(19) United States
(12) Patent Application Publication
(10) Pub. No.: US 2024/0278018 A1
(43) Pub. Date: Aug. 22, 2024

(54) STIMULATION MODULATION TO MITIGATE COLLATERAL NERVE RECRUITMENT EFFECTS

(71) Applicant: Medtronic, Inc., Minneapolis, MN (US)

(72) Inventors: Erik J. Peterson, Fridley, MN (US); Andrew J. Cleland, St. Paul, MN (US)

(21) Appl. No.: 18/545,899

(22) Filed: Dec. 19, 2023

Related U.S. Application Data
(60) Provisional application No. 63/485,740, filed on Feb. 17, 2023.

(51) Int. Cl. A61N 1/36

(52) U.S. Cl. CPC A61N 1/36139; A61N 1/3615; A61N 1/36171; A61N 1/36178

(57) ABSTRACT

A system includes a memory and processing circuitry coupled to the memory. The processing circuitry is configured to cause stimulation circuitry to deliver an electrical stimulation signal to a target neural population, wherein the electrical stimulation signal is modulated between a first level and a second level over time, wherein the delivery of the electrical stimulation signal at the first level recruits a first collateral neural population, and the delivery of the electrical stimulation signal at the second level recruits a second collateral neural population.

This application claims the benefit of U.S. Provisional Patent Application No. 63/485,740, filed Feb. 17, 2023, the entire contents of which is incorporated herein by reference.

### TECHNICAL FIELD

This disclosure relates to medical device systems and, more particularly, to medical device systems for delivery of electrical stimulation therapy.

### BACKGROUND

Electrical stimulation therapy includes delivery of electrical signals (e.g., pulses) to one or more target nerves. There are various examples of target nerves, such as peripheral or cranial nerves, nerves along the spinal cord, and nerves inside the brain. Examples of cranial and peripheral nerves include the hypoglossal or sacral nerves and may relate to facial movement or fecal/bladder function as a few examples. Other examples of peripheral nerves include those related to pain management, such as the ilioinguinal, suprascapular, axillary, femoral, and other nerves. Electrical stimulation therapy can be used for various purposes. Electrical stimulation therapy delivered to the hypoglossal nerve can cause muscle contraction that moves a tongue of a patient forward for obstructive sleep apnea (OSA) therapy. Electrical stimulation therapy delivered to nerves along the spinal cord, or some peripheral nerves, provide pain therapy by masking pain signals traveling to the brain. Electrical stimulation therapy delivered to nerves related to fecal or bladder function can better control neural signals delivered to pelvic floor muscles.

### SUMMARY

The devices, systems, and techniques of this disclosure generally relate to a medical device system and methods for electrical stimulation therapy. In electrical stimulation therapy, a medical device delivers electrical stimulation signals to one or more target neural population to achieve desired therapeutic results. This disclosure describes example techniques to modulate the electrical stimulation signals in order to mitigate effects of unwanted collateral neural population recruitment.

A neural population may refer to a group of fibers having neurons in or part of one or more nerves, including the entirety of the nerve or two or more nerves. The fibers in a neural population may have different diameters or other parameters, and may have different conduction velocities that make the fibers more or less susceptible to recruitment (e.g., activation) from delivered electrical stimulation. As described in more detail, a target neural population and a collateral neural population may refer to neurons in or part of different nerves, in some examples, and may refer to neurons of the same nerve, in some examples.

The medical device may deliver the electrical stimulation signals to target neural populations (e.g., the neural populations that when recruited provide therapeutic effect), but in addition to stimulating the target neural populations, one or more additional neural populations are also stimulated. The additional neural populations that are also stimulated, in addition to the target neural population, are referred to as collateral neural populations.

In general, nerves include bundles of neuron axons, and stimulation of some of the axons may be therapeutic while others may produce side effects. An axon also called a nerve fiber, or simply fiber, is a long, slender projection of a nerve cell, or neuron, that typically conducts electrical impulses. The bundles of neuron axons, also called fibers or fiber bundles, that, when stimulated, provide therapeutic effect are called target neural population. It is possible that nearby nerves could be unintentionally stimulated depending on the intensity of the stimulation. Also, it could be a subset of fibers of a specific nerve that may end up being unintentionally stimulated. A collateral neural population refers to the neurons in the same nerve or other nerve as the target neural population that ends up being unintentionally stimulated. As fibers are projections that extend from neurons, this disclosure describes target neural populations and collateral neural populations are including neurons, where the fibers are projections from neurons.

In some cases, stimulation of a collateral neural population results in collateral neuron recruitment, and the patient may feel effects of the collateral neuron recruitment. For instance, the electrical stimulation signals may be delivering effective therapy, but the collateral neuron recruitment may cause additional sensations, which in some cases can be an uncomfortable sensation. In some examples, stimulating a collateral neural population includes stimulating non-neuronal cells like glial cells that may be result in inflammation.

In one or more examples, the medical device may modulate one or more parameters (e.g., amplitude, pulse width, and/or frequency) that define the electrical stimulation signals. The modulation of the electrical stimulation signals may not impact the efficacy of the therapy, but can impact the collateral neuron recruitment or the manner of the collateral neuron recruitment. For instance, the modulation of the electrical stimulations signals modulates, during the delivery of therapy, the size of the collateral neural population that is recruited (i.e., the number of neurons that are activated), the length of time the collateral neural population is recruited (i.e., the length of time that the neurons are activated), etc. By modulating the collateral neuron activation, the patient may not sense the effects of the collateral neuron recruitment, or the collateral neuron recruitment may not cause discomfort.

In one example, the disclosure describes a system comprising: a memory; and processing circuitry coupled to the memory and configured to: cause stimulation circuitry to deliver an electrical stimulation signal to a target neural population, wherein the electrical stimulation signal is modulated between a first level and a second level over time, wherein the delivery of the electrical stimulation signal at the first level recruits a first collateral neural population, and the delivery of the electrical stimulation signal at the second level recruits a second collateral neural population.

In one example, the disclosure describes a method for therapy delivery, the method comprising: controlling, with processing circuitry, stimulation circuitry to deliver an electrical stimulation signal to a target neural population at a first level; and controlling, with the processing circuitry, the stimulation circuitry to modulate the electrical stimulation signal from the first level to a second level over time; and controlling, with the processing circuitry, the stimulation circuitry to modulate the electrical stimulation signal from the second level to the first level over time, wherein the delivery of the electrical stimulation signal at the first level recruits a first collateral neural population, and the delivery of the electrical stimulation signal at the second level recruits a second collateral neural population.

In one example, the disclosure describes a computer-readable storage medium storing instructions thereon that when executed cause one or more processors to: control stimulation circuitry to deliver an electrical stimulation signal to a target neural population at a first level; control the stimulation circuitry to modulate the electrical stimulation signal from the first level to a second level over time; and control the stimulation circuitry to modulate the electrical stimulation signal from the second level to the first level over time, wherein the delivery of the electrical stimulation signal at the first level recruits a first collateral neural population, and the delivery of the electrical stimulation signal at the second level recruits a second collateral neural population.

In one example, the disclosure describes a system comprising: means for controlling stimulation circuitry to deliver an electrical stimulation signal to a target neural population at a first level; means for controlling the stimulation circuitry to modulate the electrical stimulation signal from the first level to a second level over time; and means for controlling the stimulation circuitry to modulate the electrical stimulation signal from the second level to the first level over time, wherein the delivery of the electrical stimulation signal at the first level recruits a first collateral neural population, and the delivery of the electrical stimulation signal at the second level recruits a second collateral neural population.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram of an implantable medical device (IMD) system for delivering obstructive sleep apnea (OSA) therapy.

FIG. 2 is a conceptual diagram of another IMD system for delivering sacral neuromodulation (SNM).

FIG. 3 is a conceptual diagram of another IMD system for delivering spinal cord stimulation (SCS).

FIG. 4 is a block diagram illustrating example configurations of implantable medical devices (IMDs) which may be utilized in the system of FIGS. 1-3.

FIG. 5 is a block diagram illustrating an example configuration of an external programmer.

FIG. 6A is a conceptual diagram illustrating an example of an electrical stimulation signal having modulation.

FIG. 6B is a conceptual diagram illustrating another example of an electrical stimulation signal having modulation.

FIGS. 7A and 7B are conceptual diagrams illustrating an example of a first and second collateral neural population that are recruited due to modulation of electrical stimulation signal.

FIG. 8 is a flowchart illustrating an example of method of operation for delivery of electrical stimulation signals.

### DETAILED DESCRIPTION

Medical devices, systems, and techniques for delivering electrical stimulation therapy having modulated electrical stimulation signals are described. For example, a medical device (e.g., an implantable medical device) delivers the modulated electrical stimulation signals to a target neural population for therapeutic effect. However, in addition to neurons of the target neural population being recruited, the electrical stimulation signals may recruit neurons of a collateral neural population (e.g., a neural population that is not meant to be stimulated). In one or more examples, the modulation of the electrical stimulation signals modulates a number of neurons of the collateral neural population that are recruited. The modulation of the number of neurons of the collateral neural population that are recruited may change the sensation that the patient feels due to the recruitment of the neurons of the collateral neural population. The modulation of the number of neurons of the collateral neural population may refer to changing how many fibers in the collateral neural population are being recruited.

For instance, if there is no modulation of the electrical stimulation signals, then the number of neurons of the collateral neural population that are recruited may be relatively constant during the time that voltage or current of the electrical stimulation signals is being delivered. For this case, the patient may have a first type of sensation due to the recruitment of the collateral neural population, where the first type of sensation may include an uncomfortable sensation. This results in an atonic sensation. However, with the modulation of the electrical stimulation signals, the number of neurons of the collateral neural population that are recruited may change during the time that voltage or current of the electrical stimulation signals is being delivered. In such cases, the patient may have a second type of sensation due to the modulated recruitment of the collateral neural population. This results in a non-atonic sensation. In some examples, the second type of sensation may be no sensation, minimal sensation, or tolerable sensation.

In one or more examples, the modulation of the electrical stimulation signals may be suprathreshold therapeutic modulation. That is, processing circuitry may modulate an electrical stimulation signal between a first level and a second level over time. The term “level” generally refers to characteristic of the electrical stimulation signal indicative of the energy of the electrical stimulation signal. The term “level” may refer to the energy, amplitude, pulse width, or frequency. As described in more detail, the first level and the second level may also refer to electrode selection. The threshold may correspond to a therapeutic threshold, perception threshold, or any other threshold related to nerve activity.

The first level may correspond to a therapeutic threshold, and the second level is greater than the first level. For example, an amplitude of the electrical stimulation signal may be 2 mA, where at 2 mA the patient experiences therapeutic benefit from the electrical stimulation signal. During stimulation delivery (e.g., delivery of voltage or current), the processing circuitry may modulate the amplitude of the electrical stimulation signal between 2 mA and 3 mA, where 3 mA is greater than 2 mA. Accordingly, during the stimulation delivery, the modulated electrical stimulation signal remains therapeutic because the range of modulation is always at or above the therapeutic threshold.

In this disclosure, amplitude modulation is provided as one example, and the example techniques should not be considered limiting. Other examples of modulation include frequency modulation and pulse width modulation. Another example of modulation includes modulating delivery of the electrical stimulation signal between two different stimulation electrodes. For instance, the processing circuitry may cause a first electrode to deliver stimulation, and then a second electrode, and toggle delivery of the electrical stimulation signal between the first and second electrodes. Accordingly, examples of delivery of an electrical stimulation signal to a target neural population, where the electrical stimulation signal is modulated between a first level and a second level over time include amplitude, pulse width, and frequency modulation, and also include toggling between electrodes (e.g., a first level refers to stimulation from a first electrode and a second level refers to stimulation from a second electrode). In some examples, the first level is based on patient feedback (e.g., to achieve therapy). In some examples, a rate of modulation between the first level and the second level is based on patient feedback (e.g., to achieve a more comfortable mitigation in the changes in the neuron recruitment).

In this manner, by delivering an electrical stimulation signal to a target neural population that is modulated between a first level and a second level over time, there may be a change in the number of neurons of a collateral neural population that are recruited (e.g., activated). In other words, the modulation of the electrical stimulation signal causes a modulation in the collateral neural population activation. For instance, the delivery of the electrical stimulation signal at the first level recruits (e.g., activates) a first collateral neural population, and delivery of the electrical stimulation signal at the second level recruits (e.g., activates) a second collateral neural population. The first collateral neural population includes a first number of neurons, and the second collateral neural population includes a second number of neurons.

In one or more examples, the second number of neurons is greater than the first number of neurons. In some examples, the first collateral neural population may include zero number of neurons. That is, at the first level, it may be possible that no collateral neural population is recruited, and in such examples, the first collateral neural population may be zero collateral neural population.

This modulation in the nerve activation (e.g., recruitment) of different collateral neural populations (e.g., due to different numbers of neurons being recruited) may impact the manner in which the patient senses the effect of the collateral neural recruitment as compared to if there is no modulation in the neural activation. For instance, the patient may not experience discomfort due to the collateral neural population recruitment.

In this disclosure, the modulation of the electrical stimulation signal may refer to modulation that is occurring over the instance during which a voltage or current is being delivered and an electrical field is being generated. For instance, the modulation of the electrical stimulation signal may be considered as a modulation of the electrical field during the stimulation signal of a single pulse having a single polarity (e.g., the portion of the electrical stimulation signal during which an electrical field is being generated).

As another example, the modulation of the electrical stimulation signal may be considered as the level of the electrical pulses that form the electrical stimulation signal being stepped up or stepped down as a periodic wave at a particular frequency. For instance, processing circuitry (e.g., one or more processors an implantable medical device (IMD), circuitry of a stimulation generator of the IMD, or any combination of circuitry) may cause stimulation circuitry to deliver the electrical stimulation signal at the first level that recruits a first collateral neural population, and to deliver the electrical stimulation signal at the second level that recruits a second collateral neural population. As an example, the processing circuitry may generate a first electrical pulse having the first level, followed by another electrical pulse having a higher level, followed by another electrical pulse having a higher level, and so forth, until generating an Nth electrical pulse having the second level, and then generate an N+1th electrical pulse having a lower level, followed by another electrical pulse having a lower level, and so forth, until generating a N+Nth electrical pulse having the first level. These pulses may then repeat.

In this way, the electrical stimulation signal includes a repeating periodic wave of pulses, with each of the periodic wave of pulses includes a first set of plurality of pulses having levels that sequentially increase from the first level to the second level, followed by a second set of plurality of pulses having levels that sequentially decrease from the second level to the first level. The frequency of the periodic wave may be greater than 0.01 Hz, greater than 0.1 Hz, greater than 1 Hz, such as greater than 0.01 Hz, 0.1 Hz, or 1 Hz and less than or equal to 5 Hz. This periodic wave that defines the rate at which the levels of the electrical pulses of the electrical stimulation signal increase or decrease may be an envelope over the stimulation signal, and the rate at which the levels of the electrical pulses increase or decrease may be set by a frequency of the periodic wave, which may be greater than 0.01 Hz, and in some examples, between 1 Hz and 5 Hz. In general, the frequency of the periodic wave may be greater than 0.01 Hz, greater than 0.2 Hz, and so on, including greater than 1 Hz. In some examples, the frequency of the periodic wave may be less than 5 Hz, but it is possible for the frequency of the periodic wave to be greater than 5 Hz as well, including 10 Hz and 100 Hz.

As one example, processing circuitry (e.g., one or more processors of an implantable medical device (IMD), circuitry of a stimulation generator of the IMD, or any combination of circuitry) may include a mixer that mixes the electrical stimulation signal with another signal (e.g., carrier signal) that modulates the amplitude, phase, and/or frequency of the electrical stimulation signal. As another example, the processing circuitry may toggle between different electrodes (e.g., by allowing different current sources to output to respective electrodes) to modulate the electrical stimulation signal between a first level and a second level. There may be other ways in which to modulate the electrical stimulation signal.

The modulation of the electrical pulses that form the electrical stimulation signal may be different than examples in which electrical pulses that form the electrical stimulation signals are modulated for accommodation (e.g., acclimation). In accommodation, an amplitude or pulse width of the electrical stimulation signal may be periodically changed while maintaining the intensity over the different pulses. Also, the frequency at which the amplitude or pulse width are changed may be on the order of hours, days, or weeks, which is substantially less than 1 Hz or even 0.01 Hz.

Some techniques output a modified pulse periodically to evoke an action potential. However, such techniques do not include an increase and decrease in levels of sequential pulses of the electrical stimulation signal. As described above, in one or more examples, the electrical stimulation signal includes a repeating periodic wave of pulses, with each of the periodic wave of pulses includes a first set of plurality of pulses having levels that sequentially increase from the first level to the second level, followed by a second set of plurality of pulses having levels that sequentially decrease from the second level to the first level.

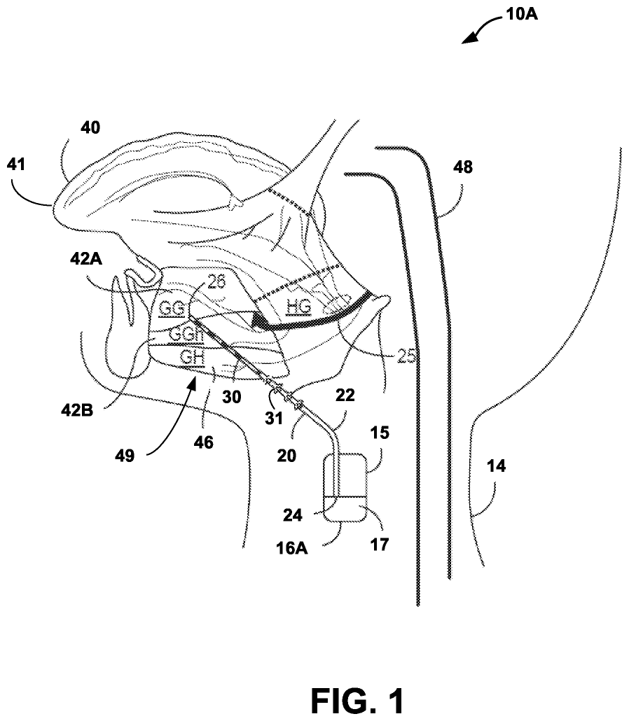

FIG. 1 is a conceptual diagram of a medical system for delivering obstructive sleep apnea (OSA) therapy. In system 10A, implantable medical device (IMD) 16A and lead 20 are implanted in patient 14. IMD 16A includes housing 15 enclosing circuitry of IMD 16A. In some examples, IMD 16A includes connector assembly 17, which is hermetically sealed to housing 15 and includes one or more connector bores for receiving a proximal end of at least one medical electrical lead 20 used for delivering OSA therapy. Although one lead 20 is illustrated in FIG. 1, there may be one or more leads 20 to which IMD 16A is coupled.

Lead 20 may include a flexible, elongated lead body 22, also called elongated member 22, that extends from lead proximal end 24 to lead distal end 26. As illustrated, lead 20 includes one or more electrodes 30 that are carried along a lead distal portion adjacent lead distal end 26 and are configured for implant within the protrusor muscles 42A, 42B, and 46 of tongue 40. As one example, the genioglossus muscle includes oblique compartment 42A and horizontal compartment 42B. In this disclosure, the genioglossus muscle is referred to as protrusor muscle 42. Protrusor muscle 46 is an example of the geniohyoid muscle.

As illustrated, distal end 26 of lead 20 includes one or more electrodes 30. Proximal end 24 of lead 20 includes one or more electrical contacts to connect to connector assembly 17. Lead 20 also includes conductors such as coils or wires that connect respective electrodes 30 to respective electrical contacts at proximal end 24 of lead 20.

While protrusor muscles 42 and 46 are described, the example techniques described in this disclosure are not limited to stimulating protrusor muscles 42 and 46. Also, FIG. 1 illustrates one set of protrusor muscles 42 and 46 (e.g., on a first side of tongue 40). The other side of tongue 40 also includes protrusor muscles. For instance, a left side of tongue 40 includes a first set of protrusor muscles 42 and 46, and a right side of tongue 40 includes a second set of protrusor muscles.

In some examples, a surgeon may implant one or more leads 20 such that one or more electrodes 30 are implanted within soft tissue, such as musculature, proximate to medial branches of one or both hypoglossal nerves. In some examples, one or more electrodes 30 may be approximately 5 mm (e.g., 2 mm to 8 mm) from a major trunk of the hypoglossal nerve. In some examples, one or more electrodes 30 may be placed in an area of protrusor muscles 42 and 46 that include motor points, where each nerve axon terminates in the muscle (also called the neuro-muscular junction). The motor points are not at one location but spread out in the protursor muscles. Leads 20 may be implanted such that one or more electrodes 30 may be generally in the area of the motor points (e.g., such that the motor points are within 1 to 10 mm from one or more electrodes 30).

Tongue 40 includes a distal end (e.g., tip of tongue 40), and electrodes 30 may be implanted proximate to root 49 of tongue 40. The surgeon may implant one or more leads 20 such that one or more electrodes are implanted proximate to root 49 of tongue 40, as illustrated in FIG. 1. For example, the location for stimulation for the genioglossus muscle 42 may be approximately 30 mm (e.g., 25 mm to 35 mm) from the symphysis of the jaw (e.g., where the genioglossus and hypoglossal muscles insert). The location for stimulation for the geniohyoid muscle 46 may be approximately 40 mm (e.g., 35 mm to 45 mm) from the symphysis. For both the genioglossus muscle 42 and the geniohyoid muscle 44, the location for stimulation may be approximately 11 mm (e.g., 7 mm to 15 mm) lateral to the midline on both the right and left sides of tongue 40 for stimulating respective hypoglossal nerves. In some examples, rather than stimulating hypoglossal nerves, the examples described in this disclosure may be configured for stimulating the motor points. Stimulating the motor points may result in indirect activation of the hypoglossal nerve, but may generally be stimulating at a different location than direct stimulation to the hypoglossal nerve. As a result, in some examples, simulation of one or more motor points may result in more precise activation of muscle fibers than may be possible with stimulation of the hypoglossal nerve itself. For ease of description, the examples are described with stimulating the hypoglossal nerves, which includes examples of stimulating the motor end point.

One or more electrodes 30 of lead 20 may be ring electrodes, segmented electrodes, partial ring electrodes or any suitable electrode configuration. Ring electrodes extend 360 degrees around the circumference of the lead body of lead 20. Segmented and partial ring electrodes each extend along an arc less than 360 degrees (e.g., 90-120 degrees) around the outer circumference of the lead body of lead 20. In this manner, multiple segmented electrodes may be disposed around the perimeter of lead 20 at the same axial position of the lead. In some examples, segmented electrodes may be useful for targeting different fibers (e.g., neurons) of the same or different nerves at respective circumferential positions with respect to the lead to generate different physiological effects (e.g., therapeutic effects), permitting stimulation to be oriented directionally. In some examples, lead 20 may be, at least in part, paddle-shaped (e.g., a “paddle” lead), and may include an array of electrodes arranged as contacts or pads on a common surface, which may or may not be substantially flat and planar.

As described above, in some examples, electrodes 30 are within musculature of tongue 40. Accordingly, one or more electrodes 30 may be “intramuscular electrodes.” Intramuscular electrodes may be different than other electrodes that are placed on or along a nerve trunk or branch, such as a cuff electrode, used to directly stimulate the nerve trunk or branch. The cuff, with the cuff electrodes, includes the cuff electrodes on one side of the cuff (e.g., the side that wraps around the nerve). The example techniques described in this disclosure are not limited to intramuscular electrodes and may be extendable to electrodes placed closer to a nerve trunk or branch of the hypoglossal nerve(s). Also, in some examples, rather than one or more electrodes 30 being “intramuscular electrodes,” one or more electrodes 30 may be implanted in connective tissue or other soft tissue proximate to the hypoglossal nerve.

In some examples, lead 20 may be configured for advancement through the soft tissue, which may include the protrusor muscle tissue, to anchor electrodes 30 in proximity to the hypoglossal nerve(s) that innervate protrusor muscles 42 and/or 46 and/or motor points that connect axons of hypoglossal nerve(s) to respective muscle fibers of protrusor muscles 42 and/or 46. However, in some examples, lead 20 may be configured for advancement through vasculature of tongue 40. As one example, a surgeon may implant lead 20 in the lingual veins near the hypoglossal nerve though venous access in the subclavian vein. In such examples, one or more electrodes 30 may be “intravascular electrodes.”

As described above, electrical stimulation therapy generated by IMD 16A and delivered via one or more electrodes 30 may activate protrusor muscles 42 and 46 to move tongue 40 forward, for instance, to promote a reduction in obstruction or narrowing of the upper airway 48 during sleep. As used herein, the term “activated” or “advanced” with regard to the electrical stimulation of protrusor muscles 42 and 46 refers to electrical stimulation that causes depolarization or an action potential of the cells of the nerve (e.g., hypoglossal nerve(s)) or stimulation at the neuro-muscular junction between the nerve and the protrusor muscles (e.g., at the motor points) innervating protrusor muscles 42 and 46 and motor points and subsequent depolarization and mechanical contraction of the protrusor muscle cells of protrusor muscles 42 and 46. In some examples, protrusor muscles 42 and 46 may be activated and advanced directly by the electrical stimulation therapy.

Protrusor muscles 42 and/or 46, on a first side of tongue 40 (e.g., the left or right side of tongue 40), may be activated by a medial branch of a first hypoglossal nerve, and the protrusor muscles, on a second side of tongue 40 (e.g., the other of the left or right side of tongue 40), may be activated by a medial branch of a second hypoglossal nerve. The medial branch of a hypoglossal nerve may also be referred to as the XIIth cranial nerve. The hyoglossus and styloglossus muscles (not shown in FIG. 1), which cause retraction and elevation of tongue 40, are activated by a lateral branch of the hypoglossal nerve.

One or more electrodes 30 may be used to deliver bilateral or unilateral stimulation to protrusor muscles 42 and 46 via the medial branch of the hypoglossal nerve or branches of the hypoglossal nerve (e.g. such as at the motor point where a terminal branch of the hypoglossal nerve interfaces with respective muscle fibers of protrusor muscles 42 and/or 46). For example, one or more electrodes 30 may be coupled to output circuitry of IMD 16A to enable delivery of electrical stimulation pulses in a manner that selectively activates the right and left protrusor muscles (e.g., in a periodic, cyclical or alternating pattern) to avoid muscle fatigue while maintaining upper airway patency. Additionally, or alternatively, IMD 16A may deliver electrical stimulation to selectively activate protrusor muscles 42 and/or 46 or portions of protrusor muscles 42 and/or 46 during unilateral stimulation of the left or right protrusor muscles.

In some examples, one lead 20 may be implanted such that one or more of electrodes 30 deliver electrical stimulation to stimulate the left hypoglossal nerve (e.g., including examples of stimulating motor points) of protrusor muscles on the left side of tongue, and therefore cause the left protrusor muscles to activate. In such examples, the electrical stimulation from one or more electrodes 30 may not be of sufficient amplitude to stimulate the right hypoglossal nerve (e.g., or motor points) of protrusor muscles on the right side of tongue and cause the right protrusor muscles to activate. In some examples, one lead 20 may be implanted such that one or more of electrodes 30 deliver electrical stimulation to stimulate the right hypoglossal nerve (e.g., including examples of stimulating motor points) of protrusor muscles on the right side of tongue, and therefore cause the right protrusor muscles to activate. In such examples, the electrical stimulation from one or more electrodes 30 may not be of sufficient amplitude to stimulate the left hypoglossal nerve (e.g., or motor points) of protrusor muscles on the left side of tongue and cause the left protrusor muscles to activate. Accordingly, in some examples, two leads like lead 20 may be implanted to stimulate each of the left and right hypoglossal nerves and/or motor points of respective protrusor muscles on the left and right side of tongue 40.

For instance, continuous stimulation may cause protrusor muscles to be continuously in an activated/advanced state. This continuous contraction may cause protrusor muscles 42 and/or 46 to fatigue. In such cases, due to fatigue, the stimulation may not cause protrusor muscles 42 and/or 46 to maintain an activated/advanced state (or higher intensity of the electrical stimulation may be needed to cause protrusor muscles 42 and/or 46 to remain in the activated/advanced state). By stimulating one set of protrusor muscles (e.g., left or right), a second set (e.g., other of left or right) of protrusor muscles can be at rest. Stimulation may then alternate to stimulate the protrusor muscles that were at rest and thereby maintain protrusion of tongue 40, while permitting the protrusor muscles 42 and/or 46 that were previously activated to rest. Hence, by cycling between alternate stimulation of the left and right protrusor muscles, tongue 40 can remain in the activated/advanced state, while one of the first or second set of protrusor muscles is at rest. In addition, it may be possible for both the right and the left side of tongue 40 to be stimulated for at least a portion of the time.

Lead proximal end 24 includes a connector (not shown in FIG. 1) that may be coupled to connector assembly 17 of IMD 16A to provide electrical connection between circuitry enclosed by the housing 15 of IMD 16A. Lead body 22 encloses electrical conductors extending from each of one or more electrodes 30 to the proximal connector at proximal end 24 to provide electrical connection between output circuitry of IMD 16A and the electrodes 30.

FIG. 1 illustrates the location of IMD 16A as being within or proximate to the neck of patient 14. However, IMD 16A may be implanted in various other locations. As one example, the surgeon may implant IMD 16A in the left or right pectoral region. For instance, the surgeon may plan on implanting IMD 16A in the left pectoral region unless another medical device is already implanted in the left pectoral region. If another medical device is already implanted in the left pectoral region, the surgeon may then implant IMD 16A in the right pectoral region. There may other locations where the surgeon may implant IMD 16A such as the back of patient 14, and other locations in the head such as below and behind an ear, or in the chin, etc. The example techniques are not limited to any particular implant location of IMD 16A.

System 10A may also include an external device. The external device may be an example of a computing device. In some examples, the external device may be a clinician programmer or patient programmer. In some examples, the external device may be a device for inputting information relating to a patient. In some examples, the external device may be a wearable communication device, with a therapy request input integrated into a key fob or a wristwatch, handheld computing device, smart phone, computer workstation, or networked computing device. The external device may include a user interface that is configured to receive input from a user (e.g., patient 14, a patient caretaker or a clinician). In some examples, the user interface includes, for example, a keypad and a display, which may for example, be a liquid crystal display (LCD) or light emitting diode (LED) display. In some examples, the user interface may include a turnable knob or a representation of a turnable knob. The keypad may take the form of an alphanumeric keypad or a reduced set of keys associated with particular functions. The external device may additionally or alternatively include a peripheral pointing device, such as a mouse, via which a user may interact with the user interface. In some examples, a display of the external device may include a touch screen display, and a user may interact with the external device via the display.

A user, such as a physician, technician, surgeon, electrophysiologist, or other clinician, may also interact with the external device or another separate programmer (not shown), such as a clinician programmer, to communicate with IMD 16A. Such a user may interact with the external device to retrieve physiological or diagnostic information from IMD 16A. The user may also interact with the external device to program IMD 16A (e.g., select values for the stimulation parameter values with which IMD 16A generates and delivers stimulation and/or the other operational parameters of IMD 16A), such as magnitudes of stimulation energy, user requested periods for stimulation or periods to prevent stimulation, or any other such user customization of therapy. In some examples, the stimulation parameter values may be proposed by system 10A, for example, by a server and a user may be able to accept or reject the stimulation parameter values. In other examples, the stimulation parameter values may be set by system 10A, for example, by a server. As discussed herein, the user may also provide input to the external device.

As described above, IMD 16A may be configured to deliver stimulation (e.g., electrical stimulation signals) to the hypoglossal nerve. In this example, the hypoglossal nerve, which is an example of a peripheral nerve, may be considered as a nerve having a target neural population. The target neural population refers to neurons, such as neurons of bundles of fibers, of a nerve that when stimulated provide therapeutic effect. In some examples, the target neural population may be the entire nerve. In some examples, the target neural population may be a subset of the nerve (e.g., less than all fibers).

To achieve therapeutic effect (e.g., advancing of tongue 40 to open the airway), IMD 16A may determine the electrical stimulation signals having a therapeutic threshold amplitude, pulse width, and/or frequency or having greater than the therapeutic threshold. In some examples, the delivery of the electrical stimulation signals may activate (e.g., recruit) the target neural population, may be also recruit a collateral neural population (e.g., recruit collateral neurons). The collateral neural population may be a neural population of neurons that when stimulated (e.g., activated or recruited) result in unwanted patient sensation or results, such as inflammation. In the example of FIG. 1, examples of the target neural population include the hypoglossal nerve, and the collateral neural population includes neural population of the lingual nerve or branch of the trigeminal nerve (e.g., cranial nerve V). The lingual nerve or branch of the trigeminal nerve include sensory axons that when recruited can cause the patient to experience unintended sensations. In this example, the target neural population and the collateral neural population are in different nerves, but in some examples, the target neural population and the collateral neural population are in the same nerve.

If the amount of stimulation of the collateral neural population is consistent (e.g., there is no change the collateral neural population), the result may be an atonic sensation that can be uncomfortable or unwanted sensations. In some examples, stimulating a collateral neural population that includes non-neuronal cells like glial cells a-tonically (e.g., without modulation) may be result in inflammation.

However, if the stimulation of the collateral neural population is modulated, the result may be a non-atonic sensation that is not uncomfortable. In one or more examples, processing circuitry of IMD 16A may be configured to modulate the electrical stimulation signal between a first level and a second level over time. IMD 16A may be configured to deliver the electrical stimulation signal to the target neural population (e.g., hypoglossal nerve) where the electrical stimulation signal is modulated between the first level and the second level over time. In one or more examples, delivery of the electrical stimulation signal at the first level recruits a first collateral neural population, and the delivery of the electrical stimulation signal at the second level recruits a second collateral neural population. For instance, the first collateral neural population includes a first number of neurons, and the second collateral neural population includes a second number of neurons, and the second number of neurons is greater than the first number of neurons. Accordingly, the modulation of the electrical stimulation signal may result in the modulation of the number of neurons of the collateral neural population that are recruited, thereby causing a sensation that is not uncomfortable.

In some examples, the first collateral neural population may include zero number of neurons. That is, at the first level, it may be possible that no collateral neural population is recruited, and in such examples, the first collateral neural population may be zero collateral neural population.

Modulation of the neurons of the collateral neural population may be considered as changing the size of the collateral neural population. Accordingly, modulating the electrical stimulation signal may result in recruiting different numbers of neurons. For instance, the number of neurons of fibers that together form the collateral neural population when the stimulation is at the first level may be different than the number of neurons of fibers that together form the collateral neural population when the stimulation is at the second level. Hence, modulation of the electrical stimulation signal may be considered as modulating the size of the collateral neural population (e.g., the amount of fibers that are recruited or the amount by which fibers are recruited). That is, modulating the electrical stimulation signal between a first level and a second level may result in the delivery of the electrical stimulation signal at the first level recruiting a first collateral neural population (e.g., first number of neurons and/or fibers), and the delivery of the electrical stimulation signal at the second level recruiting a second collateral neural population (e.g., second number of neurons and/or fibers).

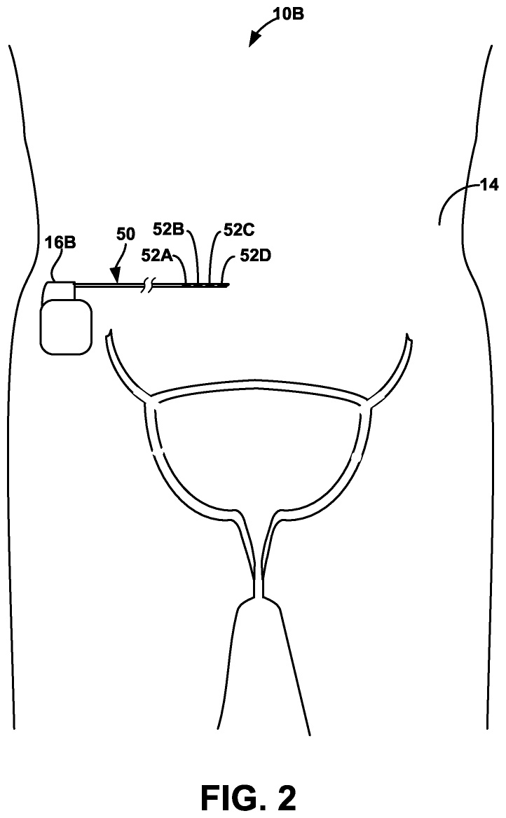

FIG. 2 is a conceptual diagram illustrating an example system 10B that includes an implantable medical device (IMD 16B) in the form of a neurostimulation device configured to deliver sacral neuromodulation (SNM), an external programmer, and one or more sensing devices in accordance with one or more techniques of this disclosure. Although described for SNM, the example techniques are also applicable for other nerves associated with pelvic disorder, such as the tibial nerve, and for providing tibial neuromodulation (TNM). The sacral nerve and tibial nerve are also examples of peripheral nerves. For TNM, IMD 16B may not be implanted as illustrated in FIG. 2, and may instead be implanted near the ankle of patient 14. For ease, the examples are described with respect to SNM with the understanding that the example techniques may be extended to other nerves, with other locations of IMD 16B, and other classes of IMD 16B (e.g., leadless IMD).

In some examples, system 10B may determine one or more stimulation setting(s) and manage delivery of neurostimulation to patient 14, e.g., to manage neurological disorder therapy such as pelvic disorders (e.g., bladder and/or bowel dysfunction therapy), such as retention, overactive bladder, urgency, urgency frequency, urinary incontinence, bladder incontinence, stress incontinence, nocturia, bowel incontinence, fecal incontinence, intractable constipation, irritable bowel syndrome, inflammatory bowel disease, neurogenic bowel and bladder (tremor, Parkinson's disease, epilepsy, multiple sclerosis, stroke, spinal cord injury, neuropathy), sexual dysfunction, obesity, gastroparesis, pelvic pain, chronic pain, and interstitial cystitis. As shown in the example of FIG. 2, therapy system 10B includes an implantable medical device (IMD) 16B (e.g., an example medical device), which is coupled to lead 50. System 10B also includes an external device, which is configured to communicate with IMD 16B via wireless communication. System 10B may also include a server which may be one or more servers in a cloud computing environment. The server may be configured to communicate with the external device and/or IMD 16B via wireless communication through a network access point (not shown) and may be collocated with the external device or may be located elsewhere, such as in a cloud computing data center.

IMD 16B generally operates as a therapy device that delivers neurostimulation (e.g., electrical stimulation signals) to, for example, a target neural population of a target tissue site proximate a spinal nerve, a sacral nerve, a sacral nerve branch, a pudendal nerve, dorsal genital nerve, a tibial nerve, a saphenous nerve, an inferior rectal nerve, a perineal nerve, or other pelvic nerves, branches of any of the aforementioned nerves, roots of any of the aforementioned nerves, ganglia of any of the aforementioned nerves, or plexus of any of the aforementioned nerves. IMD 16B provides electrical stimulation to patient 14 by generating and delivering a programmable electrical stimulation pulse (e.g., in the form of electrical pulses or an electrical signal) to a target a therapy site near lead 50 and, more particularly, near electrodes 52A-52D (collectively referred to as “electrodes 52”) disposed proximate to a distal end of lead 50.

In the example of FIG. 2, IMD 16B is implanted for stimulating the sacral nerve with lead 50. However, the location of IMD 16B may be different based on which nerve is being stimulated. For instance, IMD 16 may be implanted near the ankle for tibial stimulation, and IMD 16B may not include leads with electrodes. Instead, the electrodes used for stimulation may be formed on IMD 16B for tibial nerve stimulation.

An implanted IMD 16B may deliver electrical stimulation to provide a therapeutic effect that reduces or eliminates a dysfunctional state such as overactive bladder. The therapeutic effect may include an inhibitory physiological response related to voiding of patient 14, such as a reduction in bladder contraction frequency by a desired level or degree (e.g., percentage), a reduction in bladder afferent firing, altering a pelvic floor muscle/nerve response and/or status such as of the external urethral sphincter (EUS), levator ani nerve, external anal sphincter, and the like.

System 10B may also include an external device. The external device may be similar to the external device described with respect to FIG. 1.

Similar to the example of FIG. 1, in one or more examples, delivery of electrical stimulation signals to a target neural population (e.g., a neural population related to pelvic muscle function) may recruit a collateral neural population. In the example of FIG. 2, examples of the collateral neural population include myelinated fibers like A-beta fibers. In some examples, modulating the recruitment of A-beta fibers may cause the sensation to the patient to transition to more acceptable sensation.

To mitigate the sensation from atonic recruitment of the collateral neural population, this disclosure describes example techniques to deliver a modulated electrical stimulation signal to a target neural population that is modulated between a first level and a second level over time. The delivery of the electrical stimulation signal at the first level recruits a first collateral neural population (e.g., having a first number of neurons and/or fibers), and the delivery of the electrical stimulation signal at the second level recruits a second collateral neural population (e.g., having a second number of neurons and/or fibers).

Modulation of the neurons of the collateral neural population so that a first number of neurons of the collateral neural population are recruited with the electrical stimulation signal at the first level, and a second number of neurons of the collateral neural population are recruited with the electrical stimulation signal at the second level may be considered as changing the size of the collateral neural population. For instance, the number of neurons that together form the collateral neural population when the stimulation is at the first level (e.g., first number of neurons) may be different than the number of neurons that together form the collateral neural population when the stimulation is at the second level (e.g., second number of neurons). Hence, modulation of the electrical stimulation signal may be considered as modulating the size of the collateral neural population (e.g., the amount of neurons or fibers that are recruited or the amount by which the neurons or fibers are recruited).

The example of FIG. 2 is described with peripheral nerves that help address issues with the pelvic floor such as bladder or fecal incontinence. However, peripheral nerve stimulation may also be used for pain. For instance, stimulation of ilioinguinal, suprascapular, axillary, femoral, etc. nerves may provide pain therapy. There may be a collateral neural population within these nerves, where atonic stimulation to such collateral neural population may be result in unwanted sensations.

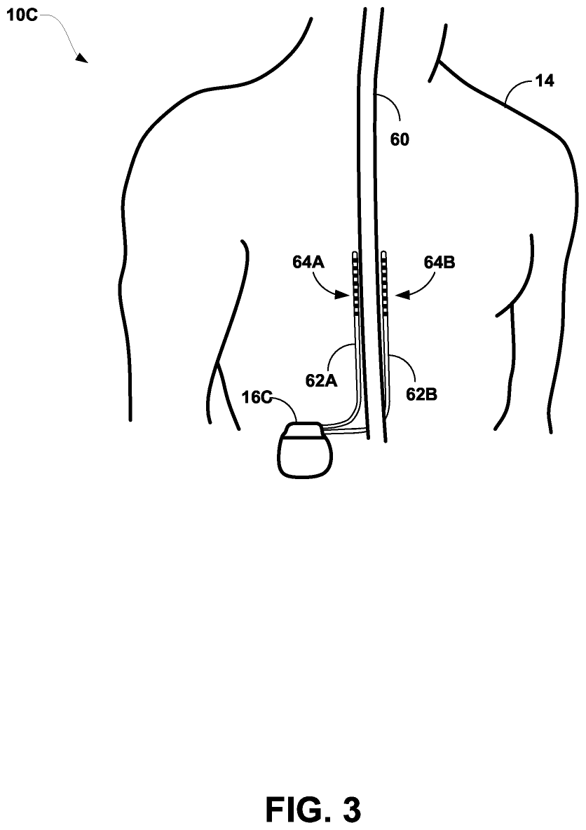

FIG. 3 is a conceptual diagram illustrating an example system 10C that includes an IMD 16C according to the techniques of the disclosure. Although the techniques described in this disclosure are generally applicable to a variety of medical devices including external devices and IMDs, application of such techniques to IMDs and, more particularly, implantable electrical stimulators (e.g., neurostimulators) will be described for purposes of illustration. More particularly, FIG. 3 will refer to an implantable spinal cord stimulation (SCS) system for purposes of illustration, but without limitation as to other types of medical devices or other therapeutic applications of medical devices.

As shown in FIG. 3, system 10C includes an IMD 16C, and leads 62A and 62B in conjunction with a patient 14. In the example of FIG. 3, IMD 16C is an implantable electrical stimulator that is configured to generate and deliver electrical stimulation therapy to patient 14 via one or more electrodes of electrodes 64A and/or 64B (collectively, “electrodes 64”) of leads 62A and/or 62B (collectively, “leads 62”), e.g., for relief of chronic pain or other symptoms. In other examples, IMD 16C may be coupled to a single lead carrying multiple electrodes or more than two leads each carrying multiple electrodes.

IMD 16C may be a chronic electrical stimulator that remains implanted within patient 14 for weeks, months, or even years. In other examples, IMD 16C may be a temporary, or trial, stimulator used to screen or evaluate the efficacy of electrical stimulation for chronic therapy. In some examples, a medical device, configured to perform techniques similar to IMD 16C, may be an external device coupled to percutaneously implanted leads. In some examples, IMD 16C uses one or more leads, while in other examples, IMD 16C is leadless.

IMD 16C may be constructed of any polymer, metal, or composite material sufficient to house the components of IMD 16C within patient 14. In this example, IMD 16C may be constructed with a biocompatible housing, such as titanium or stainless steel, or a polymeric material such as silicone, polyurethane, or a liquid crystal polymer, and surgically implanted at a site in patient 14 near the pelvis, abdomen, or buttocks. In other examples, IMD 16C may be implanted within other suitable sites within patient 14, which may depend, for example, on the target site within patient 14 for the delivery of electrical stimulation therapy. The outer housing of IMD 16C may be configured to provide a hermetic seal for components, such as a rechargeable or non-rechargeable power source. In addition, in some examples, the outer housing of IMD 16C is selected from a material that facilitates receiving energy to charge the rechargeable power source.

Electrical stimulation energy, which may be constant current or constant voltage-based pulses, for example, is delivered from IMD 16C to one or more target tissue sites of patient 14 via one or more electrodes 64 of implantable leads 62. In the example of FIG. 3, leads 62 carry electrodes 64 that are placed adjacent to the target tissue of spinal cord 60. One or more of electrodes 64 may be disposed at a distal tip of a lead 62 and/or at other positions at intermediate points along the lead. Leads 62 may be implanted and coupled to IMD 16C. Electrodes 64 may transfer electrical stimulation generated by an electrical stimulation generator in IMD 16C to tissue of patient 14. Although leads 62 may each be a single lead, lead 62 may include a lead extension or other segments that may aid in implantation or positioning of lead 62. In some examples, IMD 16C may be a leadless stimulator with one or more arrays of electrodes arranged on a housing of the stimulator rather than leads that extend from the housing. In addition, in some examples, system 10C may include one lead or more than two leads, each coupled to IMD 16C and directed to similar or different target tissue sites.

Electrodes 64 of leads 62 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 62 will be described for purposes of illustration.

The deployment of electrodes 64 via leads 62 is described for purposes of illustration, but arrays of electrodes 64 may be deployed in different ways. For example, a housing associated with a leadless stimulator may carry arrays of electrodes 64, e.g., rows and/or columns (or other patterns), to which shifting operations may be applied. Such electrodes 64 may be arranged as surface electrodes, ring electrodes, or protrusions. As a further alternative, electrode arrays may be formed by rows and/or columns of electrodes 64 on one or more paddle leads. In some examples, electrode arrays include electrode segments, which are arranged at respective positions around a periphery of a lead, e.g., arranged in the form of one or more segmented rings around a circumference of a cylindrical lead. In other examples, one or more of leads 62 are linear leads having 8 ring electrodes along the axial length of the lead. In another example, electrodes 64 are segmented rings arranged in a linear fashion along the axial length of the lead and at the periphery of the lead.

The stimulation parameter set of a stimulation program that defines the stimulation pulses of electrical stimulation therapy by IMD 16C through the electrodes of leads 62 may include information identifying which electrodes 64 have been selected for delivery of stimulation according to a stimulation program, the polarities of the selected electrodes 64, e.g., an electrode combination for the program, a voltage amplitude, a current amplitude, a pulse frequency, a pulse width, or a pulse shape of stimulation delivered by electrodes 64. These stimulation parameters values that make up the stimulation parameter set that defines pulses may be predetermined parameter values defined by a user and/or automatically determined by system 10C based on one or more factors or user input.

In some examples, lead 62 includes one or more sensors configured to allow IMD 16C to monitor one or more parameters of patient 14, such as patient activity, pressure, temperature, or other characteristics. The one or more sensors may be provided in addition to, or in place of, therapy delivery by lead 62. Rather than or in addition to lead 62 including such sensors, IMD 16C may include such sensors.

IMD 16C may be configured to deliver electrical stimulation therapy in the form of a pulse train to patient 14 via selected combinations of electrodes 64 carried by one or both of leads 62, alone or in combination with an electrode carried by or defined by an outer housing of IMD 16C. The target tissue for the electrical stimulation therapy may be any tissue affected by electrical stimulation, which may be in the form of electrical stimulation pulses or continuous waveforms. In some examples, the target tissue includes nerves, smooth muscle, or skeletal muscle. In the example illustrated by FIG. 3, the target tissue is tissue proximate spinal cord 60, such as within an intrathecal space or epidural space of spinal cord 60, or, in some examples, adjacent nerves that branch off spinal cord 60.

Leads 64 may be introduced into spinal cord 60 in via any suitable region, such as the thoracic, cervical, or lumbar regions. Stimulation of spinal cord 60 may, for example, prevent pain signals from traveling through spinal cord 60 and to the brain of patient 14. Patient 14 may perceive the interruption of pain signals as a reduction in pain and, therefore, efficacious therapy results.

IMD 16C may be configured to generate and deliver electrical stimulation therapy to a target stimulation site (e.g., target neural population) within patient 14 via electrodes 64 of leads 62 to patient 14 according to one or more therapy stimulation programs. A therapy stimulation program may generally define pulses. A therapy stimulation program may define values for one or more parameters (e.g., a parameter set) that define an aspect of the therapy delivered by IMD 16C according to that program. For example, a therapy stimulation program that controls delivery of stimulation by IMD 16C in the form of pulses may define a voltage, a current, a pulse width, a pulse rate (e.g., a pulse frequency), an electrode combination, or a pulse shape for stimulation pulses delivered by IMD 16C according to that program.

A user, such as a clinician or patient 14, may interact with a user interface of an external programmer to program IMD 16C. The external programmer (e.g., external device) may be similar to the external programmer/device described above with respect to FIGS. 1 and 2.

Similar to the examples of FIGS. 1 and 2, in some examples, in one or more examples, delivery of electrical stimulation signals to a target neural population (e.g., a neural population of or proximate spinal cord 60) may recruit a collateral neural population (e.g., different nerves or of same nerve). The spinal cord includes a nerve root for each vertebral level that corresponds to a strip of sensation around the body called a dermatome. Within a nerve root, it may be desirable to target specific fibers (e.g., target neural population). In the example of FIG. 3, examples of the collateral neural population may neurons that are within the nerve root that are not in the target neural population.

To mitigate the sensation from atonic recruitment of the collateral neural population, this disclosure describes example techniques to deliver an electrical stimulation signal to a target neural population, where the electrical stimulation signal is modulated between a first level and a second level over time. The delivery of the electrical stimulation signal at the first level recruits a first collateral neural population (e.g., having a first number of neurons and/or fibers), and the delivery of the electrical stimulation signal at the second level recruits a second collateral neural population (e.g., having a second number of neurons and/or fibers). Recruiting a first number of neurons of the collateral neural population and recruiting a second number of neurons of the collateral neural population may refer to modulating the number of neurons that are not in the target neural population that are recruited.

FIGS. 1-3 illustrate example systems for delivery of electrical stimulation signals (e.g., pulses) as part of the electrical stimulation therapy to target neural populations, in which case neurons of collateral neural populations may also be recruited. The examples of FIGS. 1-3 should not be considered exclusive to one another. That is, IMDs 16A, 16B, and 16C may be substantially similar IMDs, but programmed differently to deliver therapy to different locations within patient 14. In some examples, IMDs 16A, 16B, and 16C may be different IMDs with specialized circuitry for delivery of therapy to specific locations.

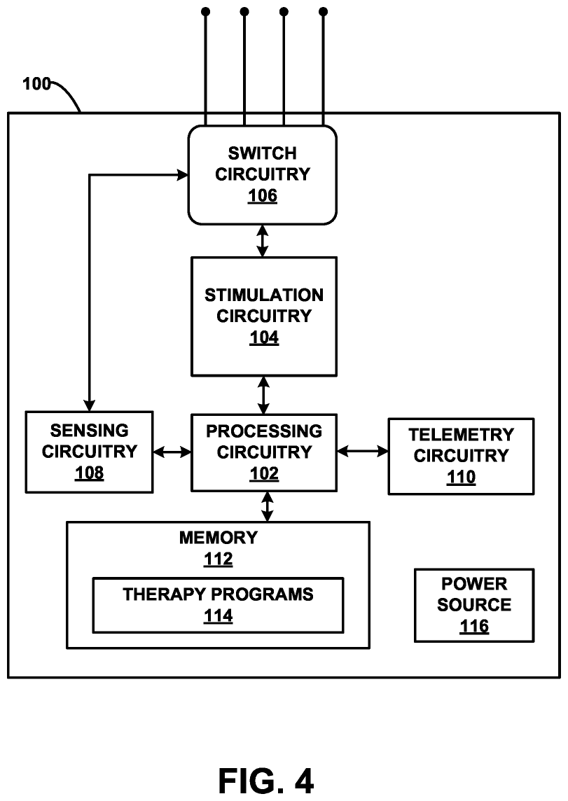

FIG. 4 is block diagram illustrating example configurations of implantable medical devices (IMDs) which may be utilized in the systems of FIGS. 1-3. As shown in FIG. 4, IMD 100. IMD 100 is an example of any of IMDs 16A, 16B, and 16C.

As illustrate, IMD 100 includes processing circuitry 102, stimulation circuitry 104, switch circuitry 106, sensing circuitry 108, telemetry circuitry 110, memory 112 for storing therapy programs 114, and power source 116. IMD 100 may include a greater or fewer number of components.

Switch circuitry 108 may be configured to, in response to instructions from processing circuitry 102, switch the coupling of electrodes (e.g., electrodes 30, 52, or 64) between sensing circuitry 108 and stimulation circuitry 104. For instance, switch circuitry 106 may be configured to allow stimulation circuitry 104 to deliver electrical stimulation signals via the one or more electrodes, and allow sensing circuitry 108 to sense a signal generated by patient 14.

In some examples, stimulation circuitry 104 may include a plurality of regulated current sources or sinks, with each current source or sink coupled to one of the electrodes. In such examples, stimulation circuitry 104 may control each current source or sink and switching between electrodes may not be necessary for therapy delivery since each one of the electrodes is individually controllable.

Although not shown in FIG. 4, in some examples, IMD 100 may include one or more sensors configured to sense posture or position of patient 14. For example, IMD 100 may include accelerometer to determine if patient 14 is lying down, whether lying on a back, whether lying on a side, and generally posture of patient 14. Another example of the one or more sensors is a motion sensor. Additional examples of the sensors include acoustical sensors or a microphone.

In some examples, the electrodes may be used to sense electromyogram (EMG) signals. Sensing circuitry 108 may be coupled to the electrodes via switch circuitry 106 to be used as EMG sensing electrodes when the electrodes are not being used for stimulation. Sensing of EMG signals is one example, and sensing circuitry 108 may be configured to sense other types of signals as well.

In general, IMD 100 may comprise any suitable arrangement of hardware, alone or in combination with software and/or firmware, to perform the techniques attributed to IMD 100. In various examples, processing circuitry 102 may include one or more processors, such as one or more microprocessors, digital signal processors (DSPs), application specific integrated circuits (ASICs), field programmable gate arrays (FPGAs), or any other equivalent integrated or discrete logic circuitry, as well as any combinations of such components.

The various units of IMD 100, such as processing circuitry 102, may be implemented as fixed-function circuits, programmable circuits, or a combination thereof. Fixed-function circuits refer to circuits that provide particular functionality, and are preset on the operations that can be performed. Programmable circuits refer to circuits that can be programmed to perform various tasks, and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, one or more of the units may be distinct circuit blocks (fixed-function or programmable), and in some examples, one or more of the units may be integrated circuits.

IMD 100 may include arithmetic logic units (ALUs), elementary function units (EFUs), digital circuits, analog circuits, and/or programmable cores, formed from programmable circuits. In examples where the operations of IMD 100 are performed using software executed by the programmable circuits, memory 112 may store the instructions (e.g., object code) of the software that processing circuitry 102 receives and executes, or another memory within IMD 100 (not shown) may store such instructions.

IMD 100 also, in various examples, may include a memory 112, such as random access memory (RAM), read only memory (ROM), programmable read only memory (PROM), erasable programmable read only memory (EPROM), electronically erasable programmable read only memory (EEPROM), flash memory, comprising executable instructions for causing the one or more processors to perform the actions attributed to them. Moreover, although sensing circuitry 108, processing circuitry 102, stimulation circuitry 102, switch circuitry 106, and telemetry circuitry 110 are described as separate circuitry, in some examples, sensing circuitry 108, processing circuitry 102, stimulation circuitry 104, switch circuitry 106, and telemetry circuitry 110 are functionally integrated. In some examples, sensing circuitry 108, processing circuitry 102, stimulation circuitry 104, switch circuitry 106, and telemetry circuitry 110 correspond to individual hardware units, such as ASICs, DSPs, FPGAs, or other hardware units.

Memory 112 stores therapy programs 114 (also called stimulation programs 114) that specify stimulation parameter values for the electrical stimulation provided by IMD 100. Memory 112 may also store instructions for execution by processing circuitry 102, in addition to stimulation programs 114. Information related to sensed parameters of patient 14 (e.g., from sensing circuitry 108 or the one or more sensors of IMD 100) may be recorded for long-term storage and retrieval by a user, and/or used by processing circuitry 102 for adjustment of stimulation parameters (e.g., amplitude, pulse width, and pulse rate). In some examples, memory 112 includes separate memories for storing instructions, electrical signal information, and stimulation programs 114. In some examples, processing circuitry 102 may select new stimulation parameters for a stimulation program 114 or new stimulation program from stimulation programs 114 to use in the delivery of the electrical stimulation based on patient input and/or monitored physiological states after termination of the electrical stimulation.

Generally, stimulation circuitry 104 generates and delivers electrical stimulation under the control of processing circuitry 102. In some examples, processing circuitry 102 controls stimulation circuitry 104 by accessing memory 112 to selectively access and load at least one of therapy programs 114 to stimulation circuitry 104. For example, in operation, processing circuitry 102 may access memory 112 to load one of stimulation programs 114 to cause stimulation circuitry 104 to operate in a particular manner.

A clinician or patient 14 may select a particular one of stimulation programs 114 from a list using a programming device, such as a patient programmer or a clinician programmer. Processing circuitry 102 may receive the selection via telemetry circuitry 110. Stimulation circuitry 104 delivers the electrical stimulation to patient 14 according to the selected program for an extended period of time, such as minutes or hours. For example, processing circuitry 102 may control switch circuitry 106 to couple the electrodes to stimulation circuitry 104.

Stimulation circuitry 104 delivers electrical stimulation according to stimulation parameters. In some examples, stimulation circuitry 104 delivers electrical stimulation in the form of electrical pulses. In such examples, relevant stimulation parameters may include a voltage or current pulse amplitude, a pulse rate, a pulse width, a duty cycle, and/or the combination of electrodes that stimulation circuitry 104 uses to deliver the stimulation signal. In some examples, stimulation circuitry 104 delivers electrical stimulation in the form of continuous waveforms. In such examples, relevant stimulation parameters may include a voltage or current amplitude, a frequency, a shape of the stimulation signal, a duty cycle of the stimulation signal, or the combination of electrodes stimulation circuitry 104 uses to deliver the stimulation signal.

In some examples, processing circuitry 102 may control stimulation circuitry 104 to deliver or terminate the electrical stimulation based on patient input received via telemetry circuitry 110. Telemetry circuitry 110 includes any suitable hardware, firmware, software or any combination thereof for communicating with another device, such as an external programmer. Under the control of processing circuitry 102, telemetry circuitry 110 may receive downlink telemetry (e.g., patient input) from and send uplink telemetry (e.g., an alert) to a programmer with the aid of an antenna, which may be internal and/or external. Processing circuitry 102 may provide the data to be uplinked to the programmer and the control signals for telemetry circuitry 110 and receive data from telemetry circuitry 110.

Generally, processing circuitry 102 controls telemetry circuitry 110 to exchange information with a medical device programmer and/or another device external to IMD 100. Processing circuitry 102 may transmit operational information and receive stimulation programs or stimulation parameter adjustments via telemetry circuitry 110. Also, in some examples, IMD 100 may communicate with other implanted devices, such as stimulators, control devices, or sensors, via telemetry circuitry 110.

Power source 116 delivers operating power to the components of IMD 100. Power source 116 may include a battery and a power generation circuit to produce the operating power. In some examples, the battery may be rechargeable to allow extended operation. Recharging may be accomplished through proximal inductive interaction between an external charger and an inductive charging coil within IMD 100. In other examples, an external inductive power supply may transcutaneously power IMD 100 whenever electrical stimulation is to occur.

In one or more examples, stimulation circuitry 106 may deliver an electrical stimulation signal (e.g., one that includes stimulation pulses). If the therapy parameters of the electrical stimulation signal result in electrical signal energy that is greater than a threshold, there is a possibility that in addition to recruiting (e.g., activating) neurons in a target neural population (e.g., the neural population to which therapy is delivered for therapeutic effect), the electrical stimulation signal recruits neurons in a collateral neural population. The recruitment of neurons in a collateral neural population may result in a sensation that patient 14 perceives, and can be uncomfortable. In some examples, recruiting a collateral neural population that includes non-neuronal cells like glial cells may be result in inflammation.

For instance, if the same number of neurons in a collateral neural population are being recruited during delivery of the electrical stimulation signal, the patient may perceive an atonic sensation. However, if the number of neurons in the collateral neural population that are being recruited changes over time (e.g., the size, shape, of recruitment level of the collateral neural population changes over time), such as during the delivery of the stimulation, the sensation that the patient perceives changes. For example, if the number of neurons in the collateral neural population is modulated, the sensation that the patient perceives may change from being uncomfortable to a more neutral sensation.

This disclosure describes examples of modulating the electrical stimulation signal delivered to a target neural population between a first level and a second level over time. By modulating the electrical stimulation signal, the collateral neural population may be modulated between a first collateral neural population having a first number of neurons and a second collateral neural population having a second number of neurons. In such examples, the delivery of the electrical stimulation signal at the first level recruits a first number of neurons of the first collateral neural population, and the delivery of the electrical stimulation signal at the second level recruits a second number of neurons of the second collateral neural population.

In some examples, it may be possible that the first number of neurons in the first collateral neural population is zero. That is, the first level does not recruit any collateral neural population, and the first collateral neural population may be considered as a zero neural population. In such examples, the electrical stimulation signal may be modulated such that there is no recruitment collateral neural population, followed by recruitment of the second collateral neural population with the second number of neurons, followed by no recruitment of a collateral neural population, followed by recruitment of the second collateral neural population with the second number of neurons, and so forth.

For example, stimulation circuitry 104 may include a mixer that mixes an electrical stimulation signal with a carrier signal, and generates a pulse having modulation, where the modulation modulates the electrical stimulation signal between a first level and a second level. Stimulation circuitry 104 may output the modulated electrical stimulation signal to a target neural population. Use of a mixer is one example, and the example techniques should not be considered limited. It may be possible to program on a pulse-by-pulse basis without using a mixer.

As described above, “first level” and “second level” may refer to a measure of characteristic of the energy of the electrical stimulation signal. For instance, first level and second level may refer to first energy level and second energy level, first amplitude level and second amplitude level, first pulse width level and second pulse width level, first frequency level and second frequency level, or any combination thereof. In some examples, the first level and the second level may refer to stimulation delivered from different electrodes.

In one or more examples, stimulation circuitry 104 may deliver the electrical stimulation signal to a target neural population (e.g., the various examples described in FIGS. 1-3) having modulation that modulates the electrical stimulation signal between a first level and a second level over time. The target neural population may a nerve along spinal cord 60 or a peripheral nerve such as hypoglossal nerve or other nerves like nerves related to bladder or fecal incontinence. As described, the delivery of the electrical stimulation signal at the first level recruits a first number of neurons of a collateral neural population, and the delivery of the electrical stimulation signal at the second level recruits a second number of neurons of the collateral neural population.

There may be various example ways in which processing circuitry 102 may cause stimulation circuitry 104 deliver the modulated electrical stimulation signal. As one example, the electrical stimulation signal includes a plurality of pulses. A pulse may be rectangular pulse having a pulse width and an amplitude. The pulse width and amplitude may together or separately define the “level” of the pulse. In one or more examples, stimulation circuitry 104 may deliver the plurality of pulses with increasing (e.g., sequentially increasing) levels until a maximum level (e.g., the second level) is reached, and then deliver the plurality of pulses with decreasing (e.g., sequentially decreasing) levels until a minimum level (e.g., the first level) is reached. The rate at which stimulation circuitry 104 increases the level or decreases the level may be defined by a periodic wave (e.g., square wave, sinusoid, triangular wave, etc.), such as a frequency of the periodic wave.

For example, if the frequency of the periodic wave is 1 Hz, then within 500 milliseconds, the level of the electrical pulses should increase from the minimum to the maximum, and from the maximum back to the minimum. For instance, the level of the electrical pulses may increase from the minimum (e.g., a first level) to the maximum (e.g., a second level) by approximately (e.g., ±10-15%) 250 milliseconds, and from the maximum (e.g., the second level) to the minimum (e.g., the first level) after approximately another 250 milliseconds, and repeat.

Accordingly, the electrical stimulation signal may include a repeating periodic wave of pulses, where each periodic wave of pulses includes a first set of plurality of pulses having levels that sequentially increase from the first level (e.g., minimum) to the second level (e.g., maximum), followed by a second set of plurality of pulses having levels that sequentially decrease from the second level (e.g., maximum) to the first level (e.g., minimum). In some examples, the frequency of the periodic wave at which the plurality of pulses of the stimulation signal increase or decrease is greater than 1 Hz. For instance, the frequency of the periodic wave is greater than 1 Hz and less than or equal to 5 Hz. The frequency of the periodic wave may be greater than 0.01 Hz.

The electrical stimulation signal having the modulation may still provide therapeutic effect. In one or more examples, the modulation may not necessarily cause the electrical stimulation signal to be at such a level that the therapeutic effect is overly impacted. As one example, the first level of the modulation corresponds to a therapeutic threshold, and the second level of the modulation may be greater than the first level. In such examples, the second number of neurons may be greater than the first number of neurons.

For instance, if electrical stimulation signal were delivered without modulation at a level (e.g., energy level based on the amplitude, pulse width, and frequency) that corresponds to a therapeutic threshold, there is a chance that neurons of a collateral neural population would be recruited (e.g., activated). In one or more examples, the modulation of the electrical stimulation signal may modulate the electrical stimulation signal such that the electrical stimulation signal remains suprathreshold for therapy. Therefore, even the electrical stimulation signal having the modulation may recruit neurons in the collateral neural population. However, because the electrical stimulation signal is modulated, the number of neurons that are recruited in the collateral neural population also modulates (e.g., the size of the collateral neural population modulates). This modulation in the number of neurons that are recruited can change the manner in which patient 14 perceives the stimulation, and may result in a less uncomfortable sensation or less inflammation as compared to stimulation with an electrical stimulation signal without modulation.

In some examples, it may be possible that electrical stimulation signals that stimulation circuitry 104 does not have sufficient energy to recruit enough neurons in a collateral neural population to cause discomfort while providing therapeutic stimulation. In such cases, modulation of the electrical stimulation signal may not be necessary.

In one or more examples, the amount of modulation needed to change the sensation of the recruitment of the neurons of the collateral neural population may be relatively low. For instance, the modulation of the electrical stimulation signal may be between a first level and a second level. In some examples, the second level may be at least 5% greater than the first level.

There may be various ways in which to determine the first level and the second level. For instance, for the first level, a clinician or user may adjust the energy of the electrical stimulation signal until patient 14 experiences therapeutic relief. For the second level, processing circuitry 102 may determine the second level based on sensed signals or based on input from patient 14.

As one example, processing circuitry 102 may receive from sensing circuitry 108 or one or more sensors from evoked neural activity from a collateral neural population or biomarkers like heart rate. The biomarker or evoked neural activity may be indicative of the first number of neurons of the first collateral neural population and/or second number of neurons of the second collateral neural population that are being recruited. As described, in some examples, the first number of neurons may be zero. For instance, processing circuitry 102 may cause stimulation circuitry 104 to deliver unmodulated electrical stimulation signals at a first level and measure the evoked neural activity or biomarker information, and then cause stimulation circuitry 104 to deliver unmodulated electrical stimulation signals a second level and measure the evoked neural activity or biomarker information. If there is a change in the evoked neural activity or the biomarker measurement, then there may be a change in the collateral neural population (e.g., change in the number of neurons of the collateral neural population) that are being recruited.

Based on the neural activity measurement or the biomarker measurements, processing circuitry 102 may be configured to receive information indicative of at least one of the first collateral neural population or the second collateral neural population. For instance, as described, processing circuitry 102 may modulate the electrical stimulation between the first level and the second level. Processing circuitry 102 may adjust one or more of the first level or the second level based on the received information to determine the second level at which to deliver stimulation. In some examples, processing circuitry 102 may adjust one or more electrodes used to deliver the electrical stimulation signal as the way to modulate the electrical stimulation signal. For instance, as described above, in some examples, to modulate the electrical stimulation signal between the first level and the second level, processing circuitry 102 may cause stimulation circuitry 104 to toggle delivery of the electrical stimulation signal between different electrodes.

As another example way to determine the second level for the modulation of the electrical stimulation signal, processing circuitry 102 may receive feedback from patient 14. For example, processing circuitry 102 (e.g., via external programmer or some other device) may receive information from patient 14 indicative of sensation due to recruitment of neurons of the collateral nerve. Processing circuitry 102 may adjust the second level based on the received information (e.g., keep adjusting until patient 14 indicates that the sensation is no longer uncomfortable), or adjust the one or more electrodes used to deliver the electrical stimulation signal.

In one or more examples, as described above, stimulation at the first level may recruit a first collateral neural population. In some examples, based on patient feedback, processing circuitry 102 may adjust the first level to a third level. Processing circuitry may cause the stimulation circuitry to deliver a second electrical stimulation signal to the target neural population. The second electrical stimulation signal is modulated between the third level and the second level or adjusted second level over time. In this example, the delivery of the second electrical stimulation signal at the third level does not recruit any collateral neural population, and the delivery of the second electrical stimulation signal at the second level or adjusted second level recruits the second collateral neural population.

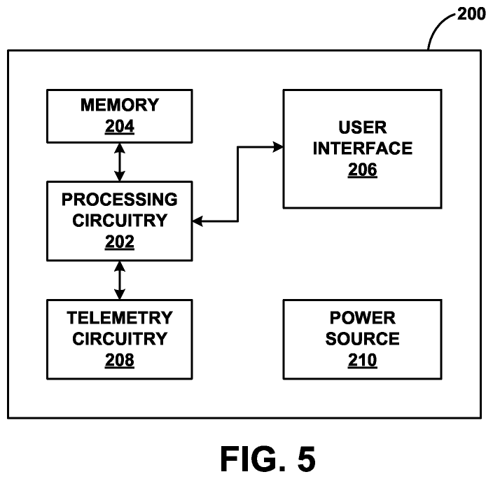

FIG. 5 is a block diagram illustrating an example configuration of an external programmer 200. While programmer 200 may generally be described as a hand-held computing device, the programmer may be a notebook computer, a cell phone, or a workstation, for example. As illustrated in FIG. 5, external programmer 200 may include processing circuitry 202, memory 204, user interface 206, telemetry circuitry 208, and power source 210.

In general, programmer 200 comprises any suitable arrangement of hardware, alone or in combination with software and/or firmware, to perform the techniques attributed to programmer 200, and processing circuitry 202, user interface 206, and telemetry circuitry 208 of programmer 200. Examples of processing circuitry 202 may include one or more processors, such as one or more microprocessors, DSPs, ASICs, FPGAs, or any other equivalent integrated or discrete logic circuitry, as well as any combinations of such components. Examples of memory 204 include RAM, ROM, PROM, EPROM, EEPROM, flash memory, a hard disk, a CD-ROM, comprising executable instructions for causing the one or more processors to perform the actions attributed to them. Moreover, although processing circuitry 202 and telemetry circuitry 208 are described as separate circuitry, in some examples, processing circuitry 202 and telemetry circuitry 208 are functionally integrated. In some examples, processing circuitry 202 and telemetry circuitry 208 correspond to individual hardware units, such as ASICs, DSPs, FPGAs, or other hardware units.

In some examples, memory 204 may further include program information (e.g., stimulation programs) defining the electrical stimulation, similar to those stored in memory 112 of IMD 100. The stimulation programs stored in memory 204 may be downloaded into memory 112 of IMD 100.

User interface 206 may include a button or keypad, lights, a speaker for voice commands, a display, such as a liquid crystal (LCD), light-emitting diode (LED), or cathode ray tube (CRT). In some examples the display may be a touch screen. As discussed in this disclosure, processing circuitry 202 may present and receive information relating to electrical stimulation and resulting therapeutic effects via user interface 206. For example, processing circuitry 202 may receive patient input via user interface 206. The input may be, for example, in the form of pressing a button on a keypad or selecting an icon from a touch screen.

Processing circuitry 202 may also present information to the patient in the form of alerts related to delivery of the electrical stimulation to patient 14 or a caregiver via user interface 206. Although not shown, programmer 200 may additionally or alternatively include a data or network interface to another computing device, to facilitate communication with the other device, and presentation of information relating to the electrical stimulation and therapeutic effects after termination of the electrical stimulation via the other device.

Telemetry circuitry 208 supports wireless communication between IMD 100 and programmer 200 under the control of processing circuitry 202. Telemetry circuitry 208 may also be configured to communicate with another computing device via wireless communication techniques, or direct communication through a wired connection. In some examples, telemetry circuitry 208 may be substantially similar to telemetry circuitry 110 of IMD 100 described above, providing wireless communication via an RF or proximal inductive medium. In some examples, telemetry circuitry 208 may include an antenna, which may take on a variety of forms, such as an internal or external antenna.

Examples of local wireless communication techniques that may be employed to facilitate communication between programmer 200 and another computing device include RF communication according to the 802.11 or Bluetooth specification sets, infrared communication (e.g., according to the IrDA standard), or other standard or proprietary telemetry protocols. In this manner, other external devices may be capable of communicating with programmer 200 without needing to establish a secure wireless connection.

Power source 210 delivers operating power to the components of programmer 200. Power source 210 may include a battery and a power generation circuit to produce the operating power. In some examples, the battery may be rechargeable to allow extended operation.

In one or more examples, processing circuitry 202 may be configured to perform at least some of the functions described in this disclosure, possibly in conjunction with processing circuitry 102. For instance, with user interface 206, processing circuitry 202 may receive information indicative of patient sensation to the modulation of the number of neurons that are recruited in patient 12. Processing circuitry 202 may determine the first level and/or second level of the modulation that is applied to the electrical stimulation signal. Processing circuitry 202 may output such information to IMD 100 (e.g., via telemetry circuitry 208). As another example, processing circuitry 202 may determine the electrodes between which the electrical stimulation signal should toggle, and may output such information to IMD 100 via telemetry circuitry 208. In general, processing circuitry 202 may be configured to perform various techniques described with respect to processing circuitry 102 based on reception of sensed signals from IMD 100 or interaction by patient 14 and/or clinician with user interface 206.

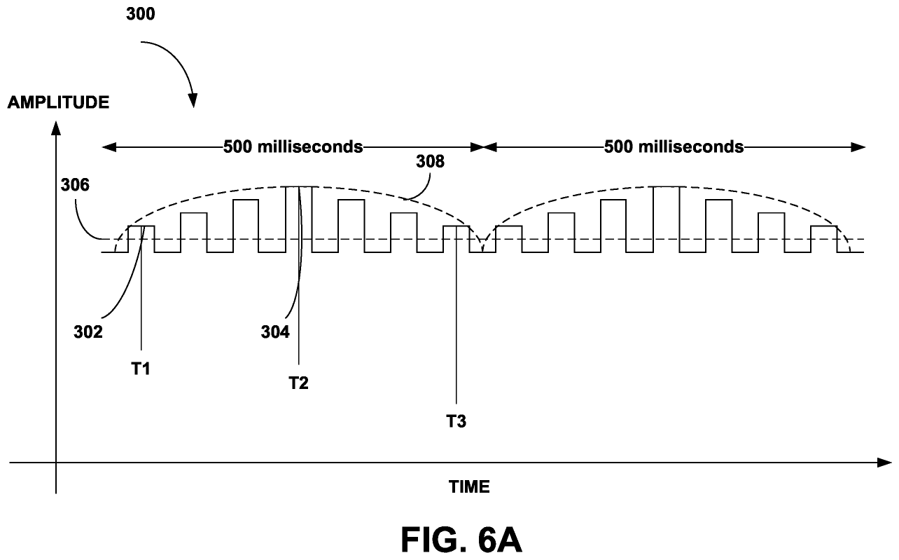

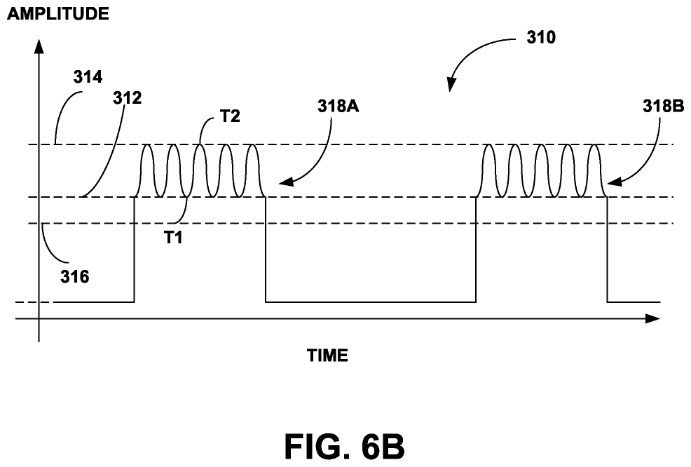

FIGS. 6A and 6B are conceptual diagram illustrating examples of an electrical stimulation signal having modulation. FIG. 6A illustrates electrical stimulation signal 300 having modulation between first level 302 and second level 304. For ease of illustration, FIG. 6A illustrates amplitude modulation on sequential pulses of electrical stimulation signal 300. However, other types of modulation may be possible such as delivery of the electrical stimulation signal by toggling the delivery of an electrical stimulation signal between a first electrode and a second electrode. In some examples, such toggling of the delivery of electrical stimulation signal between the first and second electrode may be on a pulse-by-pulse basis (e.g., a first pulse is delivered with the first electrode as anode and second electrode as cathode, and a second pulse is delivered with the second electrode as anode and second electrode as cathode).

Toggling of the delivery of electrical stimulation signal between the first and second electrode may include examples where the first and second electrodes are both cathodes or anodes, and an electrode on the housing of IMD 100 is the anode or cathode. In some examples, toggling of the delivery of electrical stimulation signal between the first and second electrodes may include examples where the first electrode is the anode, and the second electrode is the cathode, and then the first electrodes is the cathode, and the second electrode is the anode, and back-and-forth.

Referring back to FIG. 6A, as illustrated a periodic wave 308 envelopes the electrical pulses that form electrical stimulation signal 300. For instance, electrical stimulation signal 300 starts at a first level 302 and the level of the electrical stimulation signal 300 increases until electrical stimulation signal 300 reaches to a second level 304. Then, electrical stimulation 300 decrease until electrical stimulation signal 300 reaches a first level 302.

In some examples, the first level 302 is based on patient feedback (e.g., to achieve therapy). For example, the amplitude of first level 302 may be based on patient feedback indicating effective therapy. In some examples, a rate of modulation between the first level 302 and the second level 304 is based on patient feedback (e.g., to achieve a more comfortable mitigation in the changes in the neuron recruitment). That is, the rate at which electrical stimulation 300 increases from first level 302 to second level 304 or decreases from second level 304 to first level 302 may be based on patient feedback indicating comfort level. Stated another way, the time between T1 and T2 or T2 and T3 may be based on patient feedback indicating comfort level.

In one or more examples, first level 302 may correspond to a therapeutic threshold, and second level 304 is greater than the first level. In some examples, second level 304 is at least 5% greater than the first level 302. Therefore, the modulation of electrical stimulation signal 300 keeps electrical stimulation signal 300 suprathreshold for therapy. However, in FIG. 6A, line 306 represents the minimum amplitude at which neurons of a collateral neural population are recruited. That is, in FIG. 6A, to ensure therapeutic effect, electrical stimulation signal 300 may remain greater than the amplitude at which collateral neural population is recruited.

In the example of FIG. 6A, the electrical stimulation signal 300 includes a first set of plurality of pulses having levels that sequentially increase from the first level to the second level. For instance, electrical stimulation signal 300 includes the first set of plurality of pulses from time T1 to time T2 during which each of the electrical stimulation pulses (shown as square pulses) sequentially increases in amplitude form the first level 302 to the second level 304. The first set of plurality of pulses are followed by a second set of plurality of pulses (e.g., from time T2 to T3) having amplitudes that sequentially decrease from the second level 304 to the first level 302 as a periodic wave 308.

In one or more examples, periodic wave 308 (e.g., the envelope over stimulation signal 300) sets the rate at which the electrical pulses of electrical stimulation signal 300 increase or decrease. For instance, the period of periodic wave 308 is 1 second, resulting in a half period of 500 milliseconds, in FIG. 6A as an example. The frequency of periodic wave 308 may be greater than or equal to 0.01 Hz, and less than or equal to 5 Hz, but other frequency ranges are possible. As illustrated, the amplitude of the electrical stimulation signal 300 may increase at such a rate that the amplitude raises from the first level 302 to the second level 304 by time T2, and the amplitude decreases from the second level 304 to the first level 302 by time T3.

Accordingly, in FIG. 6A, the electrical stimulation signal 300 includes a repeating periodic wave 308 of pulses, each periodic wave of pulses includes a first set of plurality of pulses having levels that sequentially increase from the first level to the second level, followed by a second set of plurality of pulses having levels that sequentially decrease from the second level to the first level. A frequency of the periodic wave 308 at which the plurality of pulses of the stimulation signal 300 increase or decrease is greater than or equal to 0.01 Hz. In some examples, the frequency of the periodic wave 308 is greater than or equal to 0.01 Hz and less than or equal to 5 Hz.

In one or more examples, by modulating electrical stimulation signal 300 (e.g., between first level 302 and second level 304), the size of the collateral neural population modulates (e.g., number of neurons of the collateral neural population that are recruited also modulates), and patient discomfort may be reduced. For example, the delivery of the electrical stimulation signal 300 at the first level 302 recruits a first number of neurons of a first collateral neural population, and the delivery of the electrical stimulation signal 300 at the second level 304 recruits a second number of neurons of a second collateral neural population. In some examples, the first number of neurons is zero, and the first collateral neural population may be a zero neural population.

FIG. 6B illustrates electrical stimulation signal 310 having modulation between first level 312 and second level 314. In some examples, modulating electrical stimulation signal 310 between the first level 312 and the second level 314 may include modulation that is occurring over the instance during which a voltage or current is being delivered and an electrical field is being generated. For instance, the modulation of the electrical stimulation signal 310 may be considered as a modulation of the electrical field during the stimulation signal of a single pulse having a single polarity (e.g., the portion of the electrical stimulation signal during which an electrical field is being generated) or may be a pulse-to-pulse modulation over time of one or more stimulation parameters.

For example, as illustrated in FIG. 6B, electrical stimulation signal 310 includes first pulse 318A and second pulse 318B. As illustrated, first pulse 318A and second pulse 318A each include modulation during the pulse width of first pulse 318A and second pulse 318B (e.g., over the instance during which a voltage or current is being delivered and an electrical field is being generated). Such modulation may be over the first pulse 318A and the second pulse 318B having a single polarity.

In the example of FIG. 6B, electrical stimulation signal 310 includes a plurality of pulses (e.g., pulse 318A and pulse 318B). The first level 314 is a first amplitude of the first pulse 318A for a first time (e.g., at time T1 and other times when the amplitude is at a minimum) within the first pulse 318A. The second level 314 is a second amplitude of the first pulse 318A for a second time (e.g., at time T2 and other times when the amplitude is at a maximum) within the first pulse 318A.

In one or more examples, first level 312 may correspond to a therapeutic threshold, and second level 314 is greater than the first level. In some examples, second level 314 is at least 5% greater than the first level 312. Therefore, the modulation of electrical stimulation signal 310 keeps electrical stimulation signal 310 suprathreshold for therapy. However, in FIG. 6B, line 316 represents the minimum amplitude at which neurons of a collateral neural population are recruited. That is, in FIG. 6B, to ensure therapeutic effect, electrical stimulation signal 310 may remain greater than the amplitude at which collateral neural population is recruited.

In one or more examples, by modulating electrical stimulation signal 310 (e.g., between first level 312 and second level 314), the size of the collateral neural population modulates (e.g., number of neurons of the collateral neural population that are recruited also modulates), and patient discomfort may be reduced. For example, the delivery of the electrical stimulation signal 310 at the first level 312 recruits a first number of neurons of a first collateral neural population, and the delivery of the electrical stimulation signal 310 at the second level 314 recruits a second number of neurons of a second collateral neural population. The second number of neurons may be greater than the first number of neurons, and in some examples, the first number of neurons may be zero number of neurons.

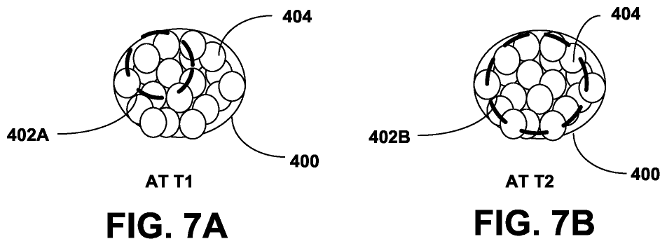

FIGS. 7A and 7B are conceptual diagrams illustrating an example of a first and second collateral neural population that are recruited due to modulation of electrical stimulation signal. For instance, FIGS. 7A and 7B illustrate nerve 400 including a plurality of neurons 404. FIG. 7A illustrates a first number of neurons 402A that form a first collateral neural population of nerve 400 that are recruited at time T1 of electrical stimulation signal 300 or 310 (e.g., when electrical stimulation signal 300 is at first level 302 or electrical stimulation signal 310 is at first level 312). FIG. 7B illustrates a second number of neurons 402B that form a second collateral neural population of nerve 400 that are recruited at time T2 of electrical stimulation signal 300 or 310 (e.g., when electrical stimulation signal 300 is at second level 304 or electrical stimulation signal 310 is at second level 314). Nerve 400 may be the same nerve that includes the target neural population, or may be a different nerve than the nerve that includes the target neural population.

As can be seen, second number of neurons 402B includes more neurons 404 than first number of neurons 402A. However, in some examples, first number of neurons 402A may be zero number of neurons. That is, first number of neurons 402A may be zero or greater than zero and less than second number of neurons 402B.

There may be various ways in which processing circuitry 102 or 202 may determine first level 302, 312 and second level 304, 314. As one example, first level 302, 312 may be based on the amplitude at which patient 14 feel therapeutic relief. In some examples, processing circuitry 102 or 202 may receive information indicative of at least one of the first number of neurons 402A or the second number of neurons 402B (e.g., based on neural activity or some other sensed signal via sensing circuitry 108). Processing circuitry 102 or 202 may adjust one or more of the second level 304, 314 based on the received information, or one or more electrodes used to deliver the electrical stimulation signal. As another example, processing circuitry 102 or 202 may receive information (e.g., via user interface 206) from patient 14 indicative of sensation due to recruitment of neurons, and adjust one or more of the second level 304, 314 based on the received information, or one or more electrodes used to deliver the electrical stimulation signal.

In one or more examples, as described above, stimulation at the first level 302, 312 may recruit a first collateral neural population. In some examples, based on patient feedback, processing circuitry 102 or 202 may adjust the first level 302, 312 to a third level. Processing circuitry 102 may also adjust the second level 304, 314, but such adjustment is not necessary. Processing circuitry 102 or 202 may cause the stimulation circuitry 104 to deliver a second electrical stimulation signal to the target neural population. The second electrical stimulation signal is modulated between the third level and the second level (or the adjusted second level) over time. In this example, the delivery of the second electrical stimulation signal at the third level does not recruit any collateral neural population, and the delivery of the second electrical stimulation signal at the second level or adjusted second level recruits the second collateral neural population.

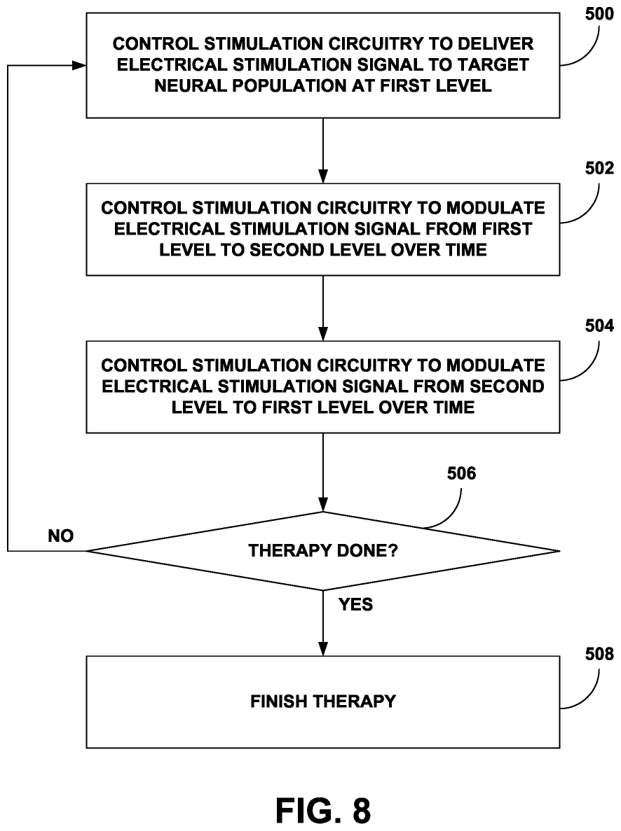

FIG. 8 is a flowchart illustrating an example of method of operation for delivery of electrical stimulation signals. In the example of FIG. 8, processing circuitry 102 may be configured to control stimulation circuitry 104 to deliver electrical stimulation signal to a target neural population at a first level (500). For example, stimulation circuitry 104 may deliver electrical stimulation signal at first level 302, 312 of FIGS. 6A and 6B, or may deliver electrical stimulation signal with a particular electrode configuration.

Processing circuitry 102 may be configured to control stimulation circuitry 104 to modulate the electrical stimulation signal from the first level to a second level over time (502). As one example, the electrical stimulation signal includes a repeating periodic wave of pulses, where each periodic wave of pulses includes a first set of plurality of pulses having levels that sequentially increase from the first level 302 to the second level 304 of FIG. 6A. As another example, the electrical stimulation signal may modulate between first level 312 and second level 314 of FIG. 6B. As another example, the electrical stimulation signal may toggle between different pairs of anode and cathode electrodes.

Processing circuitry 102 may be configured to control stimulation circuitry 104 to modulate the electrical stimulation signal from the first level to the second level over time (504). As one example, the electrical stimulation signal includes a repeating periodic wave of pulses, where each periodic wave of pulses includes a second set of plurality of pulses having levels that sequentially decrease from the second level 304 to the first level 302 of FIG. 6A. As another example, the electrical stimulation signal may modulate between second level 314 and first level 312 of FIG. 6B. As another example, the electrical stimulation signal may toggle between different pairs of anode and cathode electrodes.

Processing circuitry 102 may cause stimulation circuitry 104 to deliver modulated electrical stimulation signal to a target neural population and activate different number of neurons on a collateral neural population when delivering at the first level and the second level. For example, the delivery of the electrical stimulation signal 300, 310 at the first level 302, 312 recruits a first collateral neural population (e.g., a first number of neurons 402A). The delivery of the electrical stimulation signal 300, 310 at the second level 304, 314 recruits a second collateral neural population (e.g., a second number of neurons 402B). As illustrated in FIGS. 7A and 7B, second number of neurons 402B is greater than a first number of neurons 402A.

Processing circuitry 102 may determine whether the scheduled therapy is complete (506). For instance, the therapy delivery may be set for a certain amount of time, and may then cease. If therapy delivery is not complete (NO of 506), processing circuitry 102 may control stimulation circuitry 104 to deliver an electrical stimulation signal to a target neural population, where the electrical stimulation signal is modulated between a first level and a second level over time. The delivery of the electrical stimulation signal at the first level recruits a first collateral neural population, and the delivery of the electrical stimulation signal at the second level recruits a second collateral neural population. If therapy delivery is complete (YES of 506), processing circuitry 102 may control stimulation circuitry 104 to cease the delivery of electrical stimulation signals until some later time.

The following describes some example techniques that may be used separately or together.

Example 1. A system comprising: a memory; and processing circuitry coupled to the memory and configured to: cause stimulation circuitry to deliver an electrical stimulation signal to a target neural population, wherein the electrical stimulation signal is modulated between a first level and a second level over time, wherein the delivery of the electrical stimulation signal at the first level recruits a first collateral neural population, and the delivery of the electrical stimulation signal at the second level recruits a second collateral neural population.

Example 2. The system of example 1, wherein the target neural population, the first collateral neural population, and the second collateral neural population are in different respective nerves.

Example 3. The system of example 1, wherein the target neural population, the first collateral neural population, and the second collateral neural population are in the same nerve.

Example 4. The system of any of examples 1-3, wherein the first level corresponds to a therapeutic threshold, and the second level is greater than the first level.

Example 5. The system of any of examples 1-4, wherein the first collateral neural population includes a first number of neurons, and the second collateral neural population includes a second number of neurons, and wherein the second number of neurons is greater than the first number of neurons.

Example 6. The system of any of examples 1-5, wherein second level is at least 5% greater than the first level.

Example 7. The system of any of examples 1-5, wherein the electrical stimulation signal comprises a repeating periodic wave of pulses, each periodic wave of pulses comprising a first set of plurality of pulses having levels that sequentially increase from the first level to the second level, followed by a second set of plurality of pulses having levels that sequentially decrease from the second level to the first level.

Example 8. The system of example 7, wherein a frequency of the periodic wave at which the plurality of pulses of the stimulation signal increase or decrease is greater than or equal to 0.01 Hz.

Example 9. The system of example 8, wherein the frequency of the periodic wave is greater than or equal to 0.01 Hz and less than or equal to 5 Hz.

Example 10. The system of any of examples 1-9, wherein the processing circuitry is configured to: receive information indicative of at least one of the first collateral neural population or the second collateral neural population; and adjust one or more of: the second level based on the received information; or one or more electrodes used to deliver the electrical stimulation signal.

Example 11. The system of any of examples 1-10, wherein the processing circuitry is configured to: receive information from a patient indicative of sensation due to recruitment of neurons of at least one of the first collateral population or the second collateral population; and adjust one or more of: the second level based on the received information; or one or more electrodes used to deliver the electrical stimulation signal.

Example 12. The system of any of examples 1-11, wherein the electrical stimulation signal is a first electrical stimulation signal, and wherein the processing circuitry is configured to: adjust the first level to a third level; and cause the stimulation circuitry to deliver a second electrical stimulation signal to the target neural population, wherein the second electrical stimulation signal is modulated between the third level and the second level or adjusted second level over time, wherein the delivery of the second electrical stimulation signal at the third level does not recruit any collateral neural population, and the delivery of the second electrical stimulation signal at the second level or adjusted second level recruits the second collateral neural population.

Example 13. The system of any of examples 1-12, wherein to deliver the electrical stimulation signal to the target neural population, the processing circuitry is configured to deliver the electrical stimulation signal by toggling the delivery between a first electrode and a second electrode.

Example 14. The system of any of examples 1-13, wherein the target neural population comprises a peripheral nerve or a nerve along a spinal cord.

Example 15. The system of any of examples 1-14, wherein the target neural population comprises a hypoglossal nerve.

Example 16. The system of any of examples 1-15, wherein the system comprises an implantable medical device comprising the stimulation circuitry, the memory, and the processing circuitry.

Example 17. A method for therapy delivery, the method comprising: controlling, with processing circuitry, stimulation circuitry to deliver an electrical stimulation signal to a target neural population at a first level; and controlling, with the processing circuitry, the stimulation circuitry to modulate the electrical stimulation signal from the first level to a second level over time; and controlling, with the processing circuitry, the stimulation circuitry to modulate the electrical stimulation signal from the second level to the first level over time, wherein the delivery of the electrical stimulation signal at the first level recruits a first collateral neural population, and the delivery of the electrical stimulation signal at the second level recruits a second collateral neural population.

Example 18. The method of example 17, wherein the target neural population, the first collateral neural population, and the second collateral neural population are in different respective nerves.

Example 19. The method of example 17, wherein the target neural population, the first collateral neural population, and the second collateral neural population are in the same nerve.

Example 20. The method of any of examples 17-19, wherein the first level corresponds to a therapeutic threshold, and the second level is greater than the first level.

Example 21. The method of any of examples 17-20, wherein the first collateral neural population includes a first number of neurons, and the second collateral neural population includes a second number of neurons, and wherein the second number of neurons is greater than the first number of neurons.

Example 22. The method of any of examples 17-21, wherein second level is at least 5% greater than the first level.

Example 23. The method of any of examples 17-22, wherein controlling the stimulation circuitry to modulate the electrical stimulation signal from the first level to the second level over time, and controlling the stimulation circuitry to modulate the electrical stimulation signal from the second level to the first level over time comprises repeating a periodic wave of pulses, each periodic wave of pulses comprising a first set of plurality of pulses having levels that sequentially increase from the first level to the second level, followed by a second set of plurality of pulses having levels that sequentially decrease from the second level to the first level.

Example 24. The method of example 23, wherein a frequency of the periodic wave at which the plurality of pulses of the stimulation signal increase or decrease is greater than or equal to 0.01 Hz.

Example 25. The method of example 24, wherein the frequency of the periodic wave is greater than or equal to 0.01 Hz and less than or equal to 5 Hz.

Example 26. The method of any of examples 17-25, further comprising: receiving information indicative of at least one of the first collateral neural population or the second collateral neural population; and adjusting one or more of: the second level based on the received information; or one or more electrodes used to deliver the electrical stimulation signal.

Example 27. The method of any of examples 17-26, further comprising: receiving information from a patient indicative of sensation due to recruitment of neurons of at least one of the first collateral population or the second collateral population; and adjusting one or more of: the second level based on the received information; or one or more electrodes used to deliver the electrical stimulation signal.

Example 28. The method of any of examples 17-27, wherein controlling the stimulation circuitry to modulate the electrical stimulation signal from the first level to the second level over time, and controlling the stimulation circuitry to modulate the electrical stimulation signal from the second level to the first level over time comprises toggling the delivery of the electrical stimulation signal between a first electrode and a second electrode.

Example 29. The method of any of examples 17-28, wherein the target neural population comprises a peripheral nerve or a nerve along a spinal cord.

Example 30. The method of any of examples 17-29, wherein the target neural population comprises a hypoglossal nerve.

Example 31. A computer-readable storage medium storing instructions thereon that when executed cause one or more processors to: control stimulation circuitry to deliver an electrical stimulation signal to a target neural population at a first level; control the stimulation circuitry to modulate the electrical stimulation signal from the first level to a second level over time; and control the stimulation circuitry to modulate the electrical stimulation signal from the second level to the first level over time, wherein the delivery of the electrical stimulation signal at the first level recruits a first collateral neural population, and the delivery of the electrical stimulation signal at the second level recruits a second collateral neural population.

Example 32. The computer-readable storage medium of example 31, further comprising instructions that cause the one or more processors to perform the method of any of examples 18-30.

Example 33. A system comprising: means for controlling stimulation circuitry to deliver an electrical stimulation signal to a target neural population at a first level; means for controlling the stimulation circuitry to modulate the electrical stimulation signal from the first level to a second level over time; and means for controlling the stimulation circuitry to modulate the electrical stimulation signal from the second level to the first level over time, wherein the delivery of the electrical stimulation signal at the first level recruits a first collateral neural population, and the delivery of the electrical stimulation signal at the second level recruits a second collateral neural population.

Example 34. The system of example 33, further comprising means for performing the method of any of examples 18-30.

The techniques of this disclosure may be implemented in a wide variety of computing devices, medical devices, or any combination thereof. Any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to various modules and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated, discrete logic circuitry, or other processing circuitry, as well as any combinations of such components, remote servers, remote client devices, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. For example, any module described herein may include electrical circuitry configured to perform the features attributed to that particular module, such as fixed function processing circuitry, programmable processing circuitry, or combinations thereof.

The techniques described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

Various examples have been described herein. Any combination of the described operations or functions is contemplated. These and other examples are within the scope of the following claims.

What is claimed is:

1. A system comprising:
a memory; and
processing circuitry coupled to the memory and configured to:
cause stimulation circuitry to deliver an electrical stimulation signal to a target neural population, wherein the electrical stimulation signal is modulated between a first level and a second level over time, wherein the delivery of the electrical stimulation signal at the first level recruits a first collateral neural population, and the delivery of the electrical stimulation signal at the second level recruits a second collateral neural population.

2. The system of claim 1, wherein the target neural population, the first collateral neural population, and the second collateral neural population are in different respective nerves.

3. The system of claim 1, wherein the target neural population, the first collateral neural population, and the second collateral neural population are in the same nerve.

4. The system of claim 1, wherein the first level corresponds to a therapeutic threshold, and the second level is greater than the first level.

5. The system of claim 1, wherein the first collateral neural population includes a first number of neurons, and the second collateral neural population includes a second number of neurons, and wherein the second number of neurons is greater than the first number of neurons.

6. The system of claim 1, wherein second level is at least 5% greater than the first level.

7. The system of any of claim 1, wherein the electrical stimulation signal comprises a repeating periodic wave of pulses, each periodic wave of pulses comprising a first set of plurality of pulses having levels that sequentially increase from the first level to the second level, followed by a second set of plurality of pulses having levels that sequentially decrease from the second level to the first level.

8. The system of claim 7, wherein a frequency of the periodic wave at which the plurality of pulses of the stimulation signal increase or decrease is greater than or equal to 0.01 Hz.

9. The system of claim 8, wherein the frequency of the periodic wave is greater than or equal to 0.01 Hz and less than or equal to 5 Hz.

10. The system of claim 1, wherein the processing circuitry is configured to:
receive information indicative of at least one of the first collateral neural population or the second collateral neural population; and
adjust one or more of:
the second level based on the received information; or
one or more electrodes used to deliver the electrical stimulation signal.

11. The system of claim 1, wherein the processing circuitry is configured to:
receive information from a patient indicative of sensation due to recruitment of neurons of at least one of the first collateral neural population or the second collateral neural population; and
adjust one or more of:
the second level based on the received information; or
one or more electrodes used to deliver the electrical stimulation signal.

12. The system of claim 1, wherein the electrical stimulation signal is a first electrical stimulation signal, and wherein the processing circuitry is configured to:
adjust the first level to a third level; and
cause the stimulation circuitry to deliver a second electrical stimulation signal to the target neural population, wherein the second electrical stimulation signal is modulated between the third level and the second level or adjusted second level over time, wherein the delivery of the second electrical stimulation signal at the third level does not recruit any collateral neural population, and the delivery of the second electrical stimulation signal at the second level or adjusted second level recruits the second collateral neural population.

13. The system of claim 1, wherein to deliver the electrical stimulation signal to the target neural population, the processing circuitry is configured to deliver the electrical stimulation signal by toggling the delivery between a first electrode and a second electrode.

14. The system of claim 1, wherein the target neural population comprises a peripheral nerve or a nerve along a spinal cord.

15. The system of claim 1, wherein the target neural population comprises a hypoglossal nerve.

16. The system of claim 1, wherein the system comprises an implantable medical device comprising the stimulation circuitry, the memory, and the processing circuitry.

17. The system of claim 1, wherein at least one of:
the first level is based on patient feedback; or
a rate of modulation between the first level and the second level is based on patient feedback.

18. A method for therapy delivery, the method comprising:
controlling, with processing circuitry, stimulation circuitry to deliver an electrical stimulation signal to a target neural population at a first level;
controlling, with the processing circuitry, the stimulation circuitry to modulate the electrical stimulation signal from the first level to a second level over time; and
controlling, with the processing circuitry, the stimulation circuitry to modulate the electrical stimulation signal from the second level to the first level over time,
wherein the delivery of the electrical stimulation signal at the first level recruits a first collateral neural population, and the delivery of the electrical stimulation signal at the second level recruits a second collateral neural population.

19. The method of claim 18, wherein the target neural population, the first collateral neural population, and the second collateral neural population are in different respective nerves.

20. A computer-readable storage medium storing instructions thereon that when executed cause one or more processors to:
control stimulation circuitry to deliver an electrical stimulation signal to a target neural population at a first level;
control the stimulation circuitry to modulate the electrical stimulation signal from the first level to a second level over time; and
control the stimulation circuitry to modulate the electrical stimulation signal from the second level to the first level over time,
wherein the delivery of the electrical stimulation signal at the first level recruits a first collateral neural population, and the delivery of the electrical stimulation signal at the second level recruits a second collateral neural population.
